(19) United States
(12) Patent Application Publication
(10) Pub. No.: US 2024/0181438 A1
(43) Pub. Date: Jun. 6, 2024

(54) UNSYMMETRICAL N-HETEROCYCLIC CARBENE CATALYSTS AND METHODS USING SAME

(71) Applicant: Rutgers, The State University of New Jersey, New Brunswick, NJ (US)

(72) Inventors: Michal Szostak, Newark, NJ (US); Qun Zhao, Newark, NJ (US); Md. Mahbubur Rahman, Newark, NJ (US)

(73) Assignee: Rutgers, The State University of New Jersey, New Brunswick, NJ (US)

(21) Appl. No.: 18/278,947

(22) PCT Filed: Mar. 1, 2022
(86) PCT No.: PCT/US22/18286

§ 371 Date: Aug. 25, 2023

Related U.S. Application Data
(60) Provisional application No. 63/154,948, filed on Mar. 1, 2021.

(51) Int. Cl. B01J 31/22; B01J 31/18; B01J 37/04; C07D 233/58; C07F 1/00; C07F 1/08; C07F 5/02; C07F 15/00

(52) U.S. Cl. CPC B01J 31/2273; B01J 31/181; B01J 31/2291; B01J 37/04; C07D 233/58; C07F 1/005; C07F 1/08; C07F 5/027; C07F 15/006; B01J 2231/323; B01J 2531/16; B01J 2531/17; B01J 2531/18; B01J 2531/824

(57) ABSTRACT

The present disclosure relates in part to novel complexes of unsymmetrical N-heterocylic carbene (NHC) ligands and group 10 or 11 metals. The present disclosure further relates to methods of electrophilic functionalization of alkynes and/or nitriles using the NHC catalysts described herein.

### CROSS-REFERENCE TO RELATED APPLICATIONS

The present application claims priority under 35 U.S.C. § 119(e) to U.S. Provisional Application No. 63/154,948 filed Mar. 1, 2021, which application is hereby incorporated by reference in its entirety herein.

### STATEMENT REGARDING FEDERALLY SPONSORED RESEARCH

This invention was made with Government support under GM133326 awarded by the National Institutes of Health and CHE-1650766 awarded by the National Science Foundation. The Government has certain rights in this invention.

### BACKGROUND

Electrophilic functionalization reactions using group 11 metals represent a versatile approach for the synthesis of complex pharmaceuticals, fine chemicals, functional materials, and polymers. These reactions permit functionalization of π-systems from feedstock materials (e.g. olefins, alkynes, allenes, heterocycles, arenes, and conjugated systems) into materials useful in the synthesis of complex molecules. In general, steric and electronic requirements for N-Heterocyclic Carbene (NHC) ancillary ligands supporting Cu, Au, and Ag complexes at the +1 oxidation state are different from those of ligands utilized in traditional cross-coupling chemistry utilizing Pd⁰ or Ni⁰, which generally require for strong σ-donation and bulky wingtip substituents to facilitate oxidative addition and reductive elimination.

In contrast, the use of π-accepting NHC ligands that provide a sufficiently strong metal-carbene bond in the presence of limited steric hindrance has dominated ligand design for linear group 11 metal-NHC complexes. In this respect, IPr and IMes NHCs have been the most successful NHC ligands to date. However, modification of the sterics and/or electronics of IPr and IMes have thus far led to significant decreases in catalytic activity.

Thus, there is a need in the art for a class of NHC ligands for group 10 and 11 transition metals utilizing an alternative scaffold for more efficient transition metal catalyzed reactions, non-limiting examples including electrophilic functionalization reactions, including but not limited to, hydroboration, carboboration, and hydrolysis reactions. The present disclosure addresses and solves this need.

### BRIEF SUMMARY

In one aspect, the present disclosure provides a compound of formula (I):

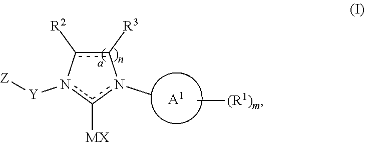

wherein R¹, R², R³, A¹, M, X, Y, Z, m, and n are defined within the scope of the present disclosure.

In another aspect, the present disclosure provides a method of promoting a reaction between an alkyne and a borylation reagent, the method comprising contacting the alkyne and the borylation reagent in the presence of a base, a protic solvent or an electrophile, and the compound of the present disclosure.

In another aspect, the present disclosure provides a method of promoting hydration of a first reagent, the method comprising contacting the first reagent and water in the presence of the compound of the present disclosure. In certain embodiments, the first reagent is an alkyne. In certain embodiments, the first reagent is a nitrile.

In another aspect, the present disclosure provides a compound of formula (III):

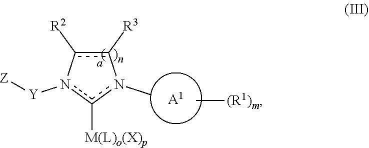

wherein R¹, R², R³, A¹, M, L, X, Y, Z, m, n, o, and p are defined within the scope of the present disclosure.

### BRIEF DESCRIPTION OF THE FIGURES

The drawings illustrate generally, by way of example, but not by way of limitation, various embodiments of the present application.

FIG. 1A provides the results of a substrate scope evaluation of alkynes in the β-hydroboration reaction using B₂(pin)₂ as the borylation reagent in the presence of either [Cu(NHC-1)Cl] or [Cu(IMes)Cl]. The catalyst loading, yield, and syn/anti B-H addition ratios are provided (e.g. 0.02%, 98%, (>98:2)) for each alkyne evaluated with [Cu(NHC-1)Cl] (top) and [Cu(IMes)Cl] (bottom). Conditions for the reaction are as follows: [Cu(NHC)Cl] (0.02-1.0 mol %), alkyne (1.0 equiv), B₂(pin)₂ (1.2 equiv), NaOH (0.05 equiv), MeOH (0.05 M), THF (0.5 M), rt, 16 h. FIG. 1B provides results for the carboboration of alkynes (i.e., 1-phenyl-1-propyne and 3-(hex-1-yn-1-yl)benzonitrile) using B₂(pin)₂ and MeI in the presence of either [Cu(NHC-1)Cl] or [Cu(IMes)Cl]. The yield and anti/syn B-H addition ratios are provided (e.g. 96%, A:B=3/97) for each alkyne evaluated for [Cu(NHC-1)Cl] (top) or [Cu(IMes)Cl] (bottom). Conditions for the reactions are as follows: [Cu(NHC)Cl] (2.0 mol %), alkyne (1.0 equiv), B₂(pin)₂ (1.2 equiv), NaOH (0.05 equiv), MeI (8 equiv), THF (0.05 M), rt, 16 h.

FIG. 2 provides the kinetic profile of the β-borylation of 1-phenyl-1-propyne using B₂(pin)₂ in the presence of either [Cu(NHC-1)Cl] (A) or [Cu(IMes)Cl] (B). Conditions for the reactions are as follows: [Cu(NHC)Cl] (0.02 mol %), alkyne (1.0 equiv), B₂(pin)₂ (1.2 equiv), NaOH (0.05 equiv), MeOH (0.05 M), THF (0.5 M), rt, 0.25-9 h.

FIG. 3 provides the results of a substrate scope evaluation of the β-hydroboration reaction of various alkynes using B₂(pin)₂ in the presence of [Ag(NHC-1)Cl]. Conditions for the reactions are as follows: [Ag(NHC-1)] (2 mol %), alkyne (1 equiv), B₂(pin)₂ (1.2 equiv), KOtBu (0.05 equiv), MeOH (0.5 M), 50 ° C., 16 h.

FIG. 4 provides the results of a substrate scope evaluation of the hydration of various alkynes using [Au(NHC-1)Cl]. The yield and turnover number (TON) are provided for each example. For the hydration of phenylacetylene, the influence of catalyst loading on TON was evaluated (100 ppm and 50 ppm), and compared to the use of [Au(IPr)Cl] at 100 ppm for comparison. Conditions for the reactions are as follows: [Au(NHC)Cl] (100 ppm), alkyne (1.0 equiv), AgSbF₆ (cat.), 1,4-dioxane/H₂O (0.5 M, 2:1), 120° C., 16 h.

FIG. 5 provides the results of a substrate scope evaluation of the hydration of nitriles using [Au(NHC-1)NTf₂] with a catalyst loading of 2 mol %. Yields reported for 2,4,6-trimethylbenzamide, 3-formylbenzamide, picolinamide, isonicotinamide, thiophene-3-carboxamide, pyrimidine-2-carboxamide, and pyrazine-2-carboxamide were obtained with a catalyst loading of 5 mol %. Conditions for the reactions are as follows: [Au(NHC-1)NTf₂] (2-5 mol %), nitrile (1 equiv), H₂O/THF (0.5 M, 1:1), 140° C., 16 h.

### DETAILED DESCRIPTION

The present disclosure relates in part to novel complexes of unsymmetrical N-heterocyclic carbenes (NHCs) with group 10 and 11 metals. The disclosure further relates to the use of the catalysts described herein as active catalysts in organic reactions. The complexes, referred to herein as unsymmetrical NHCs, may be utilized as catalysts in a broad range of reactions utilizing group 10 or 11 metals, including but not limited to hydroelement addition, element-element addition, carbo-element addition, addition, hydration, hydroamination, hydroarylation, hydroalkoxylation, hydrocarboxylation, cyclization, hydrofluorination, and hydroboration, in addition to a variety of tandem reaction processes. The synthesis, characterization, and application of several unsymmetrical NHCs (e.g. Cu(I)-NHC, Au(I)-NHC, and Ag(I)-NHC) are described herein.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃ ⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aralkynyl” as used herein refers to alkynyl groups as defined herein in which a hydrogen or carbon bond of an alkynyl group is replaced with a bond to an aryl group as defined herein. Representative aralkynyl groups include, but are not limited to, phenylacetylene and naphthylacetylene.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “borylation reagent” as used herein refers to any of a number of electrophilic boron containing species, including, but not limited to boranes, diboranes, boronic acids (RB(OH)₂), boronic esters (RB(OR)₂), or diboronic esters ((OR)₂B—B(OR)₂), which, either independently or in the presence of additional reagents and/or catalysts, are suitable to react with an alkene or alkyne.

The term “B₂(pin)₂” refers to bis(pinacolato)diboron.

The term “counter anion” as used herein refers to a negatively charged ion that accompanies a cationic species (i.e. positively charged ion) in order to maintain electric neutrality. For example, the chloride ion (Cl⁻) is the counter anion to sodium (Na⁺) in NaCl. Non-limiting examples of counter anions include F⁻, Cl⁻, Br⁻, I⁻, H⁻, N₃⁻, F₃CS(═O)₂O⁻ (OTf), (F₃CS(═O)₂)₂N⁻ (NTf₂), F₃CC(═O)O⁻ (TFA), BF₄⁻, and PF₆⁻.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “electrophile” as used herein refers to a chemical species that forms a bond with a nucleophile by accepting an electron pair in a chemical reaction (e.g. S_{N}1, S_{N}2, and carbonyl [1,2]-addition). Non-limiting examples of electrophiles include alkyl halides (e.g. MeI), benzyl halides (e.g. BnBr), dihalides (e.g. Br₂), aldehydes (e.g. Ph-CHO), acyl halides (e.g. Ph(C═O)Cl), N-electrophiles (e.g. RC(═O)ONR₂), and O-electrophiles (e.g. RC(═O)O₂R).

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaralkynyl” as used herein refers to alkynyl groups as defined herein in which a hydrogen or carbon bond of an alkynyl group is replaced with a bond to a heteroaryl group as defined herein. Representative aralkynyl groups include, but are not limited to, 2-ethynylpyridine and 2-ethynylthiophene.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f] azepine (10,11-dihydro-5H-dibenz[b,f] azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “Lewis acid” as used herein refers to a chemical species that possesses an empty orbital which is capable of accepting electrons from a Lewis base.

The term “neutral ligand” or “ligand” as used herein, refers to a ligand having no net charge prior to association with, or after dissociation from, a metal center. Non-limiting examples of neutral ligands include alkene (e.g., cyclooctadiene), CO, amine (e.g., NMe₃), phosphine (e.g., PPh₃), and pyridyl ligands, wherein coordination occurs via the nitrogen lone pair of the pyridyl group.

The term “monovalent” as used herein refers to a substituent connecting via a single bond to a substituted molecule. When a substituent is monovalent, such as, for example, F or Cl, it is bonded to the atom it is substituting by a single bond.

The term “nitrile” as used herein refers to an organic compound comprising a cyano group (C≡N).

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “protic solvent” as used herein refers to a solvent that has a hydrogen atom bound to a heteroatom such as O, N, or S, such that the H⁺ is labile. Non-limiting examples of protic solvents include methanol, ethanol, isopropanol, acetic acid, water, n-butanol, and formic acid. Conversely, an “aprotic solvent” as used herein refers to a solvent lacking dissociable hydrogen ions (i.e. non-acidic) to an appreciable extent. Non-limiting examples of aprotic solvents include ethyl acetate (EtOAc), diethyl ether (Et₂O), tetrahydrofuran (THF), dimethylformamide (DMF), and 1,4-dioxane.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “standard temperature and pressure” as used herein refers to 20° C. and 101 kPa.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” or as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

### Preparation of Compounds

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C , ¹⁴C , ³⁶Cl, ¹⁸F , ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

The compounds of the present disclosure can be prepared by the general schemes described herein, using the synthetic method known by those skilled in the art. The following examples illustrate non-limiting embodiments of the compound(s) described herein and their preparation.

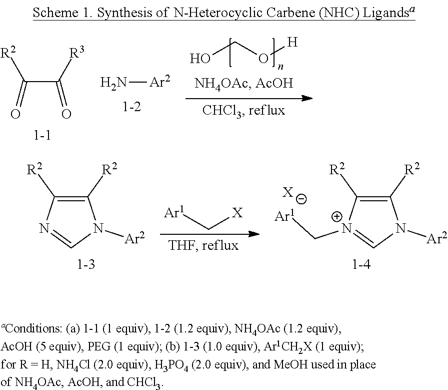

The N-heterocyclic carbene (NHC) ligands of the present disclosure were prepared according to Scheme 1, wherein R³, R⁴, Ar¹, Ar², and X are defined within the scope of the present disclosure.

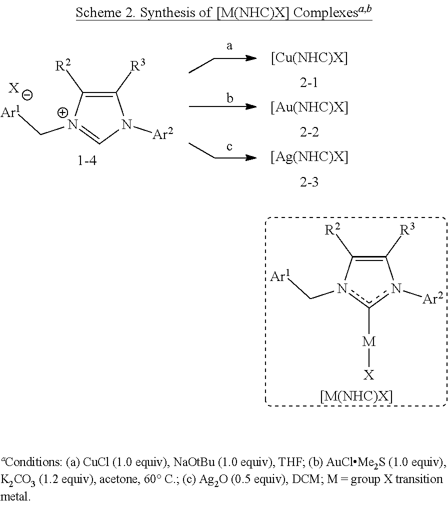

The group XI N-heterocyclic carbene (NHC) complexes of the present disclosure were prepared from the corresponding NHC ligands according to Scheme 2. Treatment of NHC 1-4 with a base in the presence of a metal salt (i.e. CuCl, AuCl, and Ag₂O) provides the corresponding NHC-metal complex (i.e. 2-1, 2-2, and 2-3, respectively). In one aspect, the NHC-metal complexes of the present disclosure are denoted as follows: [M(NHC-#)X], wherein M is a group 11 metal, NHC-# indicates the compound number of the NHC ligand, and X corresponds to the counter anion. While the conditions provided for Scheme 2 provide chloride salts of the NHC-metal complexes, alternative salts may be obtained in a number of ways, including but not limited to salt metathesis.

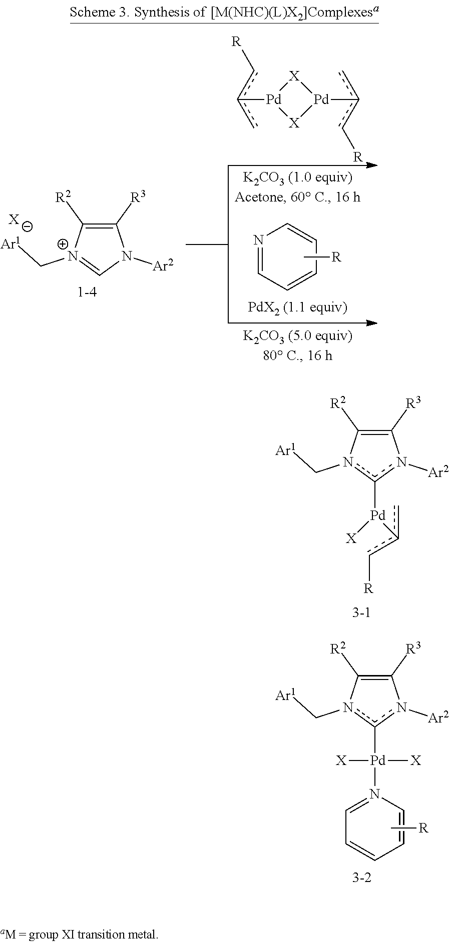

The group X N-heterocyclic carbene (NHC) complexes of the present disclosure may be prepared from the corresponding NHC ligands according to Scheme 3. Treatment of NHC 1-4 with a base in the presence of a Pd(II) dimer (e.g., [Pd(cinnamyl)Cl₂]₂) provides the corresponding π-allyl NHC-metal complex (3-1). Further, complexes of formula 3-2 may be prepared by treatment of NHC ligand 1-4 with a Pd(II) salt (e.g., PdCl₂), in the presence of an optionally substituted pyridine and a base.

In one aspect, the NHC-metal complexes of the present disclosure are denoted as follows: [M(NHC-#)(L/X)X], wherein M is a group 10 metal, NHC-# indicates the compound number of the NHC ligand, and X corresponds to a counter anion. In certain embodiments, a complex may comprise a species which is both a ligand and a counter ion (e.g., allyl), wherein the π-bond serves as a ligand and the anion serves as the counter ion.

### Compounds

The present disclosure relates in part to a compound of formula (I):

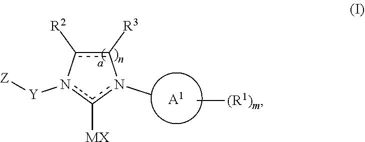

wherein:

M is a group XI (11) transition metal;

X is a counter anion;

Y is CR^{a})(R¹);

Z is selected from the group consisting of optionally substituted phenyl, optionally substituted naphthyl, optionally substituted C₄-C₁₀ heteroaryl, —C(═O)C₁-C₆-C₁₀ aryl, —C(═O)C₁-C₆ alkyl, —C(═O)OR^{a}, CN, and —C(═O)N(R^{a})(R^{b}),
wherein each optional substituent in Z is independently selected from the group consisting of halogen, hydroxy, amino, NO₂, CN, —C(═O)OR^{a}, N(R^{a})(R^{b}), C₁-C₃ alkyl, C₁-C₃ haloalkyl, C₂-C₆ alkenyl, benzyl, phenyl, and naphthyl, and C₄-C₁₀ heteroaryl;

each occurrence of [symbol] is a single or double bond,
wherein no more than one [symbol] bonding a C atom and a N atom is a double bond;

A¹ is selected from the group consisting of phenyl, naphthyl, and C₄-C₁₀ heteroaryl;

each occurrence of R¹ is independently selected from the group consisting of OR^{a}, N(R^{a})(R^{b}), optionally substituted C₁-C₆ alkyl, optionally substituted C₃-C₁₂ cycloalkyl, optionally substituted C₂-C₁₀ heterocycloalkyl optionally substituted C₂-C₆ alkenyl, optionally substituted benzyl, optionally substituted phenyl, optionally substituted naphthyl, optionally substituted C₄-C₁₀ heteroaryl,
wherein each optional substituent in R¹ is independently selected from the group consisting of C₁-C₃ alkyl, C₂-C₆ alkenyl, benzyl, phenyl, and naphthyl, and C₄-C₁₀ heteroaryl;

R² and R³ are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
R² and R³ may combine with the ring to which they are attached to form a C₄-C₁₂ cycloalkyl, C₆-C₁₀ aryl, C₄-C₁₀ heteroaryl,
wherein each optional substituent in R² and R³ is independently at least one substituent selected from the group consisting of C₁-C₃ alkyl, C₂-C₆ alkenyl, phenyl, naphthyl, C₄-C₁₀ heteroaryl, OR^{b}, N(R^{a})(R^{b}), CN, CF₃, OCF₃, C(═O)R^{b}, N(R)S(═O)₂R^{b}, C(═O)N(R^{a})(R^{b}), and C(═O)OR^{b};

each occurrence of R^{a} is independently selected from the group consisting of H, optionally substituted C₁-C₃ alkyl, optionally substituted C₃-C₁₂ cycloalkyl, optionally substituted C₂-C₁₀ heterocycloalkyl, optionally substituted C₆-C₁₀ aryl, and optionally substituted C₄-C₁₀ heteroaryl,
wherein each optional substituent in R^{a} is independently at least one substituent selected from the group consisting of C₁-C₃ alkyl, halogen, CN, NO₂, OR^{b}, OCF₃, and CF₃;

each occurrence of R^{b} is independently selected from the group consisting of H, C₁-C₆ alkyl, C₂-C₆ alkenyl, benzyl, phenyl, naphthyl, and C₄-C₁₀ heteroaryl;

m is an integer selected from the group consisting of 0, 1, 2, 3, 4, and 5; and

n is an integer which is either 1 or 2.

In certain embodiments, Y is —CH₂—.

In certain embodiments, bond a is a double bond.

In certain embodiments, m is 1. In certain embodiments, m is 2. In certain embodiments, m is 3. In certain embodiments, n is 1. In certain embodiments, n is 2.

In certain embodiments, R¹ is methyl. In certain embodiments, R¹ is ethyl. In certain embodiments, R¹ is n-propyl. In certain embodiments, R¹ is isopropyl. In certain embodiments, R¹ is n-butyl. In certain embodiments, R¹ is sec-butyl. In certain embodiments, R¹ is isobutyl. In certain embodiments, R¹ is tert-butyl. In certain embodiments, R¹ is cyclopropyl. In certain embodiments, R¹ is cyclobutyl. In certain embodiments, R¹ is cyclopentyl. In certain embodiments, R¹ is cyclohexyl. In certain embodiments, R¹ is cycloheptyl. In certain embodiments, R¹ is cyclooctyl. In certain embodiments, R¹ is —CH(CH₂CH₃)₂. In certain embodiments, R¹ is —CH(CH(CH₃)₂)₂. In certain embodiments, R¹ is —CH(CH₂CH₂CH₂CH₃)₂. In certain embodiments, R¹ is phenyl. In certain embodiments, R¹ is CHPh₂. In certain embodiments, R¹ is —OCH₃. In certain embodiments, R¹ is —O(CH(CH₃)₂). In certain embodiments, R¹ is CHPh₂.

In certain embodiments, the compound of formula (I) is a compound of formula (II):

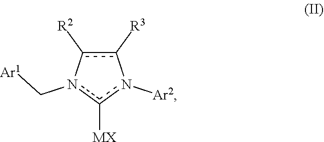

wherein:

X is selected from the group consisting of halogen, OS(═O)₂R^{c}, OC(═O)R^{c}, N(C(═O)R^{c})₂, tetracoordinate boronate, hexacoordinate phosphorus, and H;

each occurrence of R^{c} is independently selected from the group consisting of C₁-C₃ alkyl, C₁-C₃ haloalkyl, and phenyl optionally substituted with at least one substituent selected from the group consisting of C₁-C₃ alkyl and halogen;

Ar¹ and Ar² are each independently selected from the group consisting of optionally substituted phenyl, optionally substituted naphthyl, and optionally substituted C₄-C₁₀ heteroaryl,
wherein each optional substituent in Ar¹ and Ar² is independently at least one substituent selected from the group consisting of OR^{a}, N(R^{a})(R^{b}), C₁-C₁₂ alkyl, C₃-C₁₂ cycloalkyl, C₂-C₁₀ heterocycoalkyl, C₁-C₃ haloalkyl, benzyl, and —CH₂—(C₆-C₁₀ heteroaryl), and
wherein the optional benzyl and —CH₂—(C₆-C₁₀ heteroaryl) substituents in Ar¹ and Ar² are independently optionally substituted with at least one substituent selected from the group consisting of C₁-C₃ alkyl, C₆-C₁₀ aryl, and C₆-C₁₀ heteroaryl; and

R² and R³ are each independently selected from the group consisting of H, C₁-C₆ alkyl, C₂-C₆ alkenyl, phenyl, and naphthyl, C₄-C₁₀ heteroaryl.

In certain embodiments, R² is H. In certain embodiments, R² is methyl. In certain embodiments, R² is ethyl. In certain embodiments, R² is n-propyl. In certain embodiments, R² is isopropyl. In certain embodiments, R² is n-butyl. In certain embodiments, R² is sec-butyl. In certain embodiments, R² is isobutyl. In certain embodiments, R² is tert-butyl.

In certain embodiments, R³ is H. In certain embodiments, R³ is methyl. In certain embodiments, R³ is ethyl. In certain embodiments, R³ is n-propyl. In certain embodiments, R³ is isopropyl. In certain embodiments, R³ is n-butyl. In certain embodiments, R³ is sec-butyl. In certain embodiments, R³ is isobutyl. In certain embodiments, R³ is tert-butyl. In certain embodiments, R² and R³ are identical.

In certain embodiments, Ar¹ is phenyl. In certain embodiments, Ar¹ is

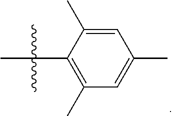 [SMILES: *c1c(C)cc(C)cc1C]

In certain embodiments, Ar¹ is

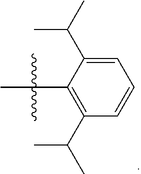 [SMILES: *c1c(C(C)C)cccc1C(C)C]

In certain embodiments, Ar¹ is

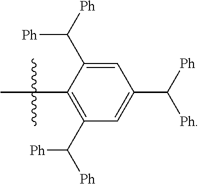 [SMILES: *c1c(C(c2ccccc2)c2ccccc2)cc(C(c2ccccc2)c2ccccc2)cc1C(c1ccccc1)c1ccccc1]

In certain embodiments, Ar¹ is

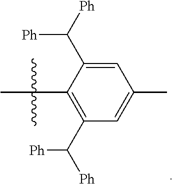 [SMILES: *c1c(C(c2ccccc2)c2ccccc2)cc(C)cc1C(c1ccccc1)c1ccccc1]

In certain embodiments, Ar² is phenyl. In certain embodiments, Ar² is

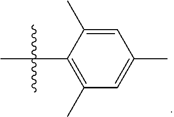 [SMILES: *c1c(C)cc(C)cc1C]

In certain embodiments, Ar² is

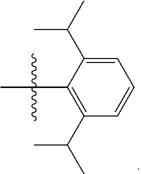 [SMILES: *c1c(C(C)C)cccc1C(C)C]

In certain embodiments, Ar² is

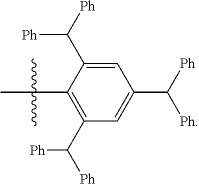 [SMILES: *c1c(C(c2ccccc2)c2ccccc2)cc(C(c2ccccc2)c2ccccc2)cc1C(c1ccccc1)c1ccccc1]

In certain embodiments, Ar² is

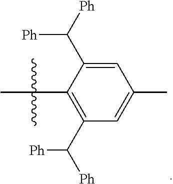 [SMILES: *c1c(C(c2ccccc2)c2ccccc2)cc(C)cc1C(c1ccccc1)c1ccccc1]

In certain embodiments, M is Cu. In certain embodiments, M is Ag. In certain embodiments, M is Au.

In certain embodiments, X is Cl. In certain embodiments, X is Br. In certain embodiments, X is triflate (OTf). In certain embodiments, X is bistriflamide (NTf₂). In certain embodiments, X is tosylate (OTs). In certain embodiments, X is trifluoroacetate (TFA). In certain embodiments, X is BF₄. In certain embodiments, X is PF₆. In certain embodiments, X is H.

In certain embodiments, the compound is 1-(2,6-diisopropylphenyl)-4,5-dimethyl-3-(2,4,6-trimethylbenzyl)-imidazol-2-ylidene copper(I) chloride. In certain embodiments, the compound is 1-(2,6-diisopropylphenyl)-3-(2,4,6-trimethylbenzyl)-imidazol-2-ylidene copper(I) chloride. In certain embodiments, the compound is 1-mesityl-4,5-dimethyl-3-(2,4,6-trimethylbenzyl)-imidazol-2-ylidene copper(I) chloride. In certain embodiments, the compound is 1-mesityl-3-(2,4,6-trimethylbenzyl)-imidazol-2-ylidene copper(I) chloride. In certain embodiments, the compound is 1-benzyl-3-(2,6-diisopropylphenyl)-4,5-dimethyl-imidazol-2-ylidene copper(I) chloride. In certain embodiments, the compound is 1-(2,6-diisopropylphenyl)-4,5-dimethyl-3-(2,4,6-trimethylbenzyl)-imidazol-2-ylidene gold(I) bistriflimide. In certain embodiments, the compound is 1-(2,6-diisopropylphenyl)-4,5-dimethyl-3-(2,4,6-trimethylbenzyl)-imidazol-2-ylidene gold(I) chloride. In certain embodiments, the compound is 1-(2,6-diisopropylphenyl)-3-(2,4,6-trimethylbenzyl)-imidazol-2-ylidene gold(I) chloride. In certain embodiments, the compound is 1-mesityl-4,5-dimethyl-3-(2,4,6-trimethylbenzyl)-imidazol-2-ylidene gold(I) chloride. In certain embodiments, the compound is 1-mesityl-3-(2,4,6-trimethylbenzyl)-imidazol-2-ylidene gold(I) chloride. In certain embodiments, the compound is 1-benzyl-3-(2,6-diisopropylphenyl)-4,5-dimethyl-imidazol-2-ylidene gold(I) chloride. In certain embodiments, the compound is 1-(2,6-diisopropylphenyl)-4,5-dimethyl-3-(2,4,6-trimethylbenzyl)-imidazol-2-ylidene silver(I) chloride. In certain embodiments, the compound is 1-(2,6-diisopropylphenyl)-3-(2,4,6-trimethylbenzyl)-imidazol-2-ylidene silver(I) chloride. In certain embodiments, the compound is 1-mesityl-4,5-dimethyl-3-(2,4,6-trimethylbenzyl)-imidazol-2-ylidene silver(I) chloride. In certain embodiments, the compound is 1-mesityl-3-(2,4,6-trimethylbenzyl)-imidazol-2-ylidene silver(I) chloride. In certain embodiments, the compound is 1-benzyl-3-(2,6-diisopropylphenyl)-4,5-dimethyl-imidazol-2-ylidene silver(I) chloride.

The present disclosure further relates to a compound of formula (III):

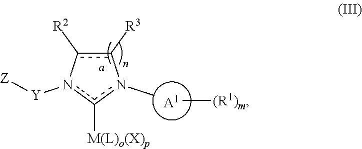

wherein:

M is a group X (10) transition metal;

L is a ligand of M, wherein each occurrence of L can be the same or different;

X is a counter anion;

Y is C(R^{a})(R^{a});

Z is selected from the group consisting of optionally substituted phenyl, optionally substituted naphthyl, optionally substituted C₄-C₁₀ heteroaryl, —C(═O)C₆-C₁₀ aryl, —C(═O)C₁-C₆ alkyl, —C(═O)OR^{a}, CN, and —C(═O)N(R^{a})(R^{b}),
wherein each optional substituent in Z is independently selected from the group consisting of halogen, hydroxy, amino, NO₂, CN, —C(═O)OR^{a}, N(R^{a})(R^{b}), C₁-C₃ alkyl, C₁-C₃ haloalkyl, C₂-C₆ alkenyl, benzyl, phenyl, and naphthyl, and C₄-C₁₀ heteroaryl;

each occurrence of [symbol] is a single or double bond,
wherein no more than one [symbol] bonding a C atom and a N atom is a double bond;

A¹ is selected from the group consisting of phenyl, naphthyl, and C₄-C₁₀ heteroaryl;

each occurrence of R¹ is independently selected from the group consisting of OR^{a}, N(R^{a})(R^{b}), optionally substituted C₁-C₁₂ alkyl, optionally substituted C₃-C₁₂ cycloalkyl, optionally substituted C₂-C₁₀ heterocycloalkyl, optionally substituted C₂-C₆ alkenyl, optionally substituted benzyl, optionally substituted phenyl, optionally substituted naphthyl, optionally substituted C₄-C₁₀ heteroaryl,
wherein each optional substituent in R¹ is independently selected from the group consisting of C₁-C₃ alkyl, C₂-C₆ alkenyl, benzyl, phenyl, and naphthyl, and C₄-C₁₀ heteroaryl;

R² and R³ are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
R² and R³ may combine with the ring to which they are attached to form a C₄-C₁₂ cycloalkyl, C₆-C₁₀ aryl, or C₄-C₁₀ heteroaryl,
wherein each optional substituent in R² and R³ is independently at least one substituent selected from the group consisting of C₁-C₃ alkyl, C₂-C₆ alkenyl, phenyl, naphthyl, C₄-C₁₀ heteroaryl, OR^{b}, N(R^{a})(R^{b}), CN, CF₃, OCF₃, C(═O)R^{b}, N(R)S(═O)₂R^{b}, C(═O)N(R^{a})(R^{b}), and C(═O)OR^{b};

each occurrence of R^{a} is independently selected from the group consisting of H, optionally substituted C₁-C₃ alkyl, optionally substituted C₃-C₁₂ cycloalkyl, optionally substituted C₂-C₁₀ heterocycloalkyl, optionally substituted C₆-C₁₀ aryl, and optionally substituted C₄-C₁₀ heteroaryl,
wherein each optional substituent in R^{a} is independently at least one substituent selected from the group consisting of C₁-C₃ alkyl, halogen, CN, NO₂, OR^{b}, OCF₃, and CF₃;

each occurrence of R^{b} is independently selected from the group consisting of H, C₁-C₆ alkyl, C₂-C₆ alkenyl, benzyl, phenyl, naphthyl, and C₄-C₁₀ heteroaryl;

m is an integer selected from the group consisting of 0, 1, 2, 3, 4, and 5;

n is an integer which is either 1 or 2;

o is an integer selected from the group consisting of 1 and 2; and

p is an integer selected from the group consisting of 1 and 2.

In certain embodiments, Y is —CH₂—.

In certain embodiments, bond a is a double bond.

In certain embodiments, m is 2 or 3.

In certain embodiments, n is 1.

In certain embodiments, o is 1.

In certain embodiments, p is 2.

In certain embodiments, R¹ is methyl. In certain embodiments, R¹ is ethyl. In certain embodiments, R¹ is n-propyl. In certain embodiments, R¹ is isopropyl. In certain embodiments, R¹ is n-butyl. In certain embodiments, R¹ is sec-butyl. In certain embodiments, R¹ is isobutyl. In certain embodiments, R¹ is tert-butyl. In certain embodiments, R¹ is cyclopropyl. In certain embodiments, R¹ is cyclobutyl. In certain embodiments, R¹ is cyclopentyl. In certain embodiments, R¹ is cyclohexyl. In certain embodiments, R¹ is cycloheptyl. In certain embodiments, R¹ is cyclooctyl. In certain embodiments, R¹ is —CH(CH₂CH₃)₂. In certain embodiments, R¹ is —CH(CH(CH₃)₂)₂. In certain embodiments, R¹ is —CH(CH₂CH₂CH₂CH₃)₂. In certain embodiments, R¹ is phenyl. In certain embodiments, R¹ is CHPh₂. In certain embodiments, R¹ is —OCH₃. In certain embodiments, R¹ is —O(CH(CH₃)₂). In certain embodiments, R¹ is CHPh₂.

In certain embodiments, the compound of formula (III) is a compound of formula (IV):

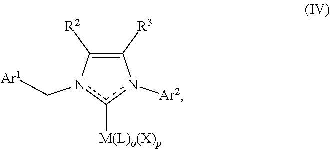

wherein:

X is selected from the group consisting of halogen, OS(═O)₂R^{c}, OC(═O)R^{c}, N(C(═O)R^{c})₂, tetracoordinate boronate, hexacoordinate phosphorus, H, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl;

L optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl;

each occurrence of R^{c} is independently selected from the group consisting of C₁-C₃ alkyl, C₁-C₃ haloalkyl, and phenyl optionally substituted with at least one substituent selected from the group consisting of C₁-C₃ alkyl and halogen;

Ar¹ and Ar² are each independently selected from the group consisting of optionally substituted phenyl, optionally substituted naphthyl, and optionally substituted C₄-C₁₀ heteroaryl,
wherein each optional substituent in Ar¹ and Ar² is independently at least one substituent selected from the group consisting of OR^{a}, N(R^{a})(R^{b}), C₁-C₁₂ alkyl, C₃-C₁₂ cycloalkyl, C₂-C₁₀ heterocycloalkyl, C₁-C₃ haloalkyl, benzyl, and —CH₂—(C₆-C₁₀ heteroaryl), and
wherein the optional benzyl and —CH₂—(C₆-C₁₀ heteroaryl) substituents in Ar¹ and Ar² are independently optionally substituted with at least one substituent selected from the group consisting of C₁-C₃ alkyl, C₆-C₁₀ aryl, and C₆-C₁₀ heteroaryl; and

R² and R³ are each independently selected from the group consisting of H, C₁-C₆ alkyl, C₂-C₆ alkenyl, phenyl, and naphthyl, C₄-C₁₀ heteroaryl.

In certain embodiments, X is Cl. In certain embodiments, X is Br. In certain embodiments, X is tosylate (OTs). In certain embodiments, X is mesylate (OMs). In certain embodiments, X is triflate (OTf). In certain embodiments, X is bistriflamide (NTf₂). In certain embodiments, X is allyl anion (i.e., vinylmethanide). In certain embodiments, X is allylbenzene anion (i.e., 3-phenylpropen-3-ide and/or 1-phenylpropen-3-ide).

In certain embodiments, L is Cl. In certain embodiments, L is 3-chloropyridine.

In certain embodiments, R² is H. In certain embodiments, R² is methyl. In certain embodiments, R² is ethyl. In certain embodiments, R² is n-propyl. In certain embodiments, R² is isopropyl. In certain embodiments, R² is n-butyl. In certain embodiments, R² is sec-butyl. In certain embodiments, R² is isobutyl. In certain embodiments, R² is tert-butyl.

In certain embodiments, R³ is H. In certain embodiments, R³ is methyl. In certain embodiments, R³ is ethyl. In certain embodiments, R³ is n-propyl. In certain embodiments, R³ is isopropyl. In certain embodiments, R³ is n-butyl. In certain embodiments, R³ is sec-butyl. In certain embodiments, R³ is isobutyl. In certain embodiments, R³ is tert-butyl. In certain embodiments, R² and R³ are identical.

In certain embodiments, Ar¹ is phenyl. In certain embodiments, Ar¹ is

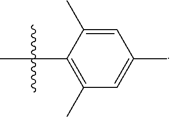 [SMILES: *c1c(C)cc(C)cc1C]

In certain embodiments, Ar¹ is

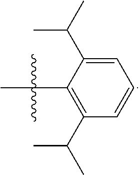 [SMILES: *c1c(C(C)C)cccc1C(C)C]

In certain embodiments, Ar² is phenyl. In certain embodiments, Ar² is

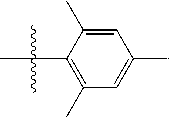 [SMILES: *c1c(C)cc(C)cc1C]

In certain embodiments, Ar² is

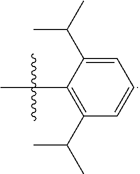 [SMILES: *c1c(C(C)C)cccc1C(C)C]

In certain embodiments, M is Pd. In certain embodiments, M is Ni. In certain embodiments, M is Pt.

In certain embodiments, the compound is allyl [1-(2,6-diisopropylphenyl)-4,5-dimethyl-3-(2,4,6-trimethylbenzyl)-1H-imidazol-2-ylidene] chloropalladium(II). In certain embodiments, the compound is cinnamyl [1-(2,6-diisopropylphenyl)-4,5-dimethyl-3-(2,4,6-trimethylbenzyl)-1H-imidazol-2-ylidene] chloropalladium(II). In certain embodiments, the compound is cinnamyl [1-mesityl-4,5-dimethyl-3-(2,4,6-trimethylbenzyl)-1H-imidazol-2-ylidene] chloropalladium(II). In certain embodiments, the compound is cinnamyl [1-(2,6-diisopropylphenyl)-3-(2,4,6-trimethylbenzyl)-1H-imidazol-2-ylidene] chloropalladium(II). In certain embodiments, the compound is cinnamyl [1-mesityl-3-(2,4,6-trimethylbenzyl)-1H-imidazol-2-ylidene] chloropalladium(II). In certain embodiments, the compound is cinnamyl [1-(2,6-diisopropylphenyl)-4,5-dimethyl-3-benzyl-1H-imidazol-2-ylidene] chloropalladium(II). In certain embodiments, the compound is 3-chloropyridine [1-(2,6-diisopropylphenyl)-4,5-dimethyl-3-(2,4,6-trimethylbenzyl)-1H-imidazol-2-ylidene] dichloropalladium(II). In certain embodiments, the compound is 3-chloropyridine [1-mesityl-4,5-dimethyl-3-(2,4,6-trimethylbenzyl)-1H-imidazol-2-ylidene] dichloropalladium(II). In certain embodiments, the compound is 3-chloropyridine [1-(2,6-diisopropylphenyl)-3-(2,4,6-trimethylbenzyl)-1H-imidazol-2-ylidene] dichloropalladium(II). In certain embodiments, the compound is 3-chloropyridine [1-mesityl-3-(2,4,6-trimethylbenzyl)-1H-imidazol-2-ylidene] dichloropalladium(II). In certain embodiments, the compound is 3-chloropyridine [1-(2,6-diisopropylphenyl)-4,5-dimethyl-3-benzyl-1H-imidazol-2-ylidene] dichloropalladium(II).

TABLE 1

| Structure | Name |
|---|---|
| 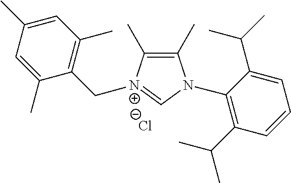 [SMILES: Cc1cc(C)c(C[n+]2cn(-c3c(C(C)C)cccc3C(C)C)c(C)c2C)c(C)c1 ; [Cl-]] | 1-(2,6-diisopropylphenyl)-4,5-dimethyl-3- (2,4,6-trimethylbenzyl)-1H-imidazol-3-ium chloride |
| 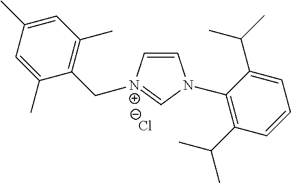 [SMILES: Cc1cc(C)c(C[n+]2ccn(-c3c(C(C)C)cccc3C(C)C)c2)c(C)c1 ; [Cl-]] | 1-(2,6-diisopropylphenyl)-3-(2,4,6- trimethylbenzyl)-1H-imidazol-3-ium chloride |
| 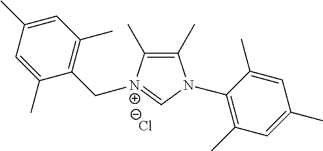 | 1-mesityl-4,5-dimethyl-3-(2,4,6- trimethylbenzyl)-1H-imidazol-3-ium chloride |
| 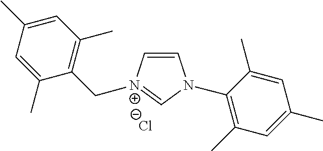 | 1-mesityl-3-(2,4,6-trimethylbenzyl)-1H- imidazol-3-ium chloride |
| 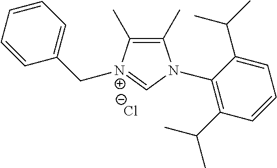 [SMILES: Cc1c(C)[n+](Cc2ccccc2)cn1-c1c(C(C)C)cccc1C(C)C ; [Cl-]] | 1-(2,6-diisopropylphenyl)-4,5-dimethyl-3- benzyl-1H-imidazol-3-ium chloride |
| 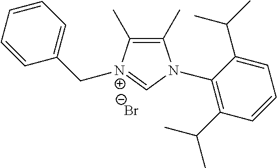 [SMILES: Cc1c(C)[n+](Cc2ccccc2)cn1-c1c(C(C)C)cccc1C(C)C ; [Br-]] | 1-(2,6-diisopropylphenyl)-4,5-dimethyl-3- benzyl-1H-imidazol-3-ium bromide |
| 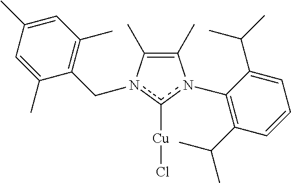 | 1-(2,6-diisopropylphenyl)-4,5-dimethyl-3- (2,4,6-trimethylbenzyl)-imidazol-2-ylidene copper(I) chloride |
| 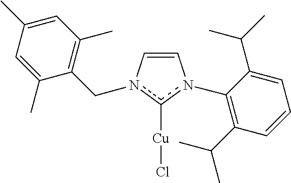 | 1-(2,6-diisopropylphenyl)-3-(2,4,6- trimethylbenzyl)-imidazol-2-ylidene copper(I) chloride |
| 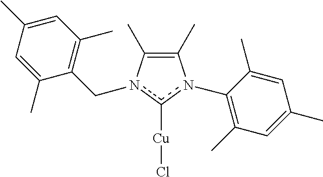 | 1-mesityl-4,5-dimethyl-3-(2,4,6- trimethylbenzyl)-imidazol-2-ylidene copper(I) chloride |
| 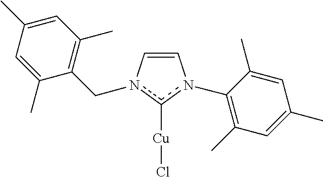 | 1-mesityl-3-(2,4,6-trimethylbenzyl)-imidazol-2- ylidene copper(I) chloride |
| 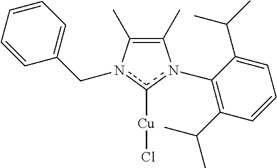 | 1-benzyl-3-(2,6-diisopropylphenyl)-4,5- dimethyl-imidazol-2-ylidene copper(I) chloride |
| 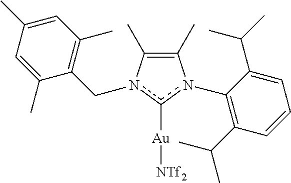 | 1-(2,6-diisopropylphenyl)-4,5-dimethyl-3- (2,4,6-trimethylbenzyl)-imidazol-2-ylidene gold(I) bistriflimide |
| 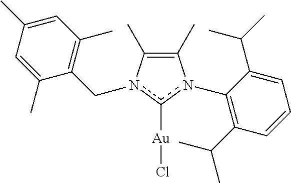 | 1-(2,6-diisopropylphenyl)-4,5-dimethyl-3- (2,4,6-trimethylbenzyl)-imidazol-2-ylidene gold(I) chloride |
| 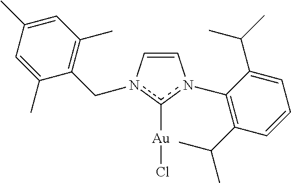 | 1-(2,6-diisopropylphenyl)-3-(2,4,6- trimethylbenzyl)-imidazol-2-ylidene gold(I) chloride |
| 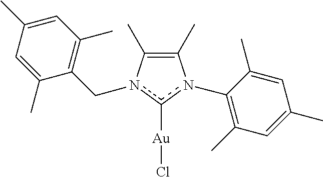 | 1-mesityl-4,5-dimethyl-3-(2,4,6- trimethylbenzyl)-imidazol-2-ylidene gold(I) chloride |
| 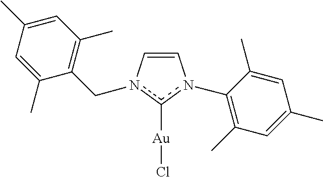 | 1-mesityl-3-(2,4,6-trimethylbenzyl)-imidazol-2- ylidene gold(I) chloride |
| 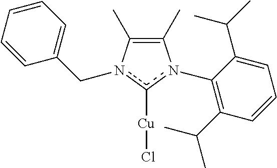 | 1-benzyl-3-(2,6-diisopropylphenyl)-4,5- dimethyl-imidazol-2-ylidene gold(I) chloride |
| 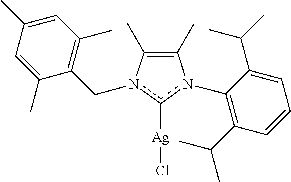 | 1-(2,6-diisopropylphenyl)-4,5-dimethyl-3- (2,4,6-trimethylbenzyl)-imidazol-2-ylidene silver(I) chloride |
| 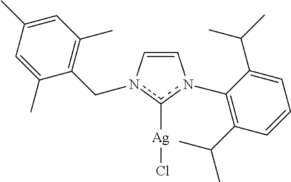 | 1-(2,6-diisopropylphenyl)-3-(2,4,6- trimethylbenzyl)-imidazol-2-ylidene silver(I) chloride |
| 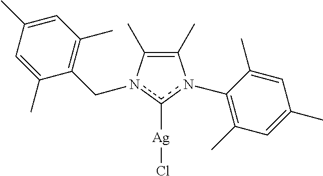 | 1-mesityl-4,5-dimethyl-3-(2,4,6- trimethylbenzyl)-imidazol-2-ylidene silver(I) chloride |
| 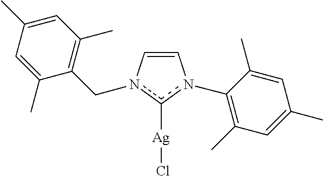 | 1-mesityl-3-(2,4,6-trimethylbenzyl)-imidazol-2- ylidene silver(I) chloride |
| 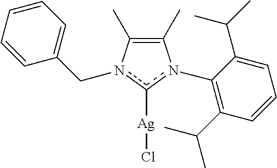 | 1-benzyl-3-(2,6-diisopropylphenyl)-4,5- dimethyl-imidazol-2-ylidene silver(I) chloride |
| 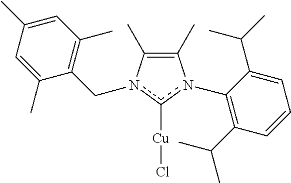 | 1-(2,6-diisopropylphenyl)-4,5-dimethyl-3- (2,4,6-trimethylbenzyl)-imidazol-2-ylidene copper(I) chloride |
| 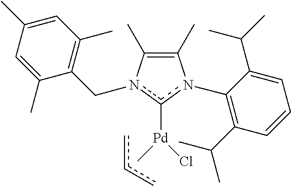 | allyl [1-(2,6-diisopropylphenyl)-4,5-dimethyl-3- (2,4,6-trimethylbenzyl)-1H-imidazol-2-ylidene] chloropalladium(II) |
| 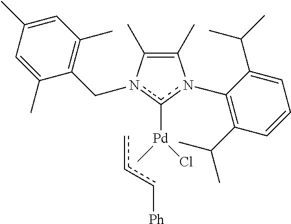 | cinnamyl [1-(2,6-diisopropylphenyl)-4,5- dimethyl-3-(2,4,6-trimethylbenzyl)-1H- imidazol-2-ylidene] chloropalladium(II) |
| 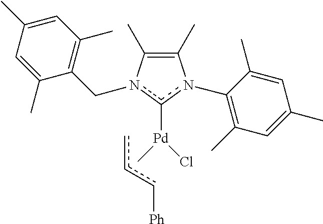 | cinnamyl [1-mesityl-4,5-dimethyl-3-(2,4,6- trimethylbenzyl)-1H-imidazol-2-ylidene] chloropalladium(II) |
| 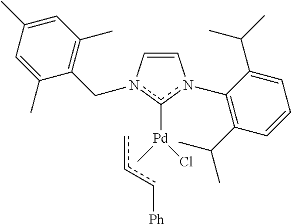 | cinnamyl [1-(2,6-diisopropylphenyl)-3-(2,4,6- trimethylbenzyl)-1H-imidazol-2-ylidene] chloropalladium(II) |
| 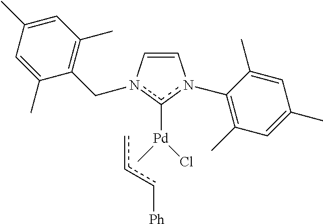 | cinnamyl [1-mesityl-3-(2,4,6-trimethylbenzyl)- 1H-imidazol-2-ylidene] chloropalladium(II) |
| 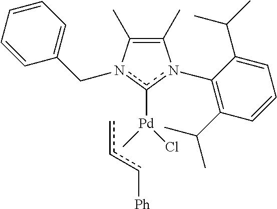 | cinnamyl [1-(2,6-diisopropylphenyl)-4,5- dimethyl-3-benzyl-1H-imidazol-2-ylidene] chloropalladium(II) |
| 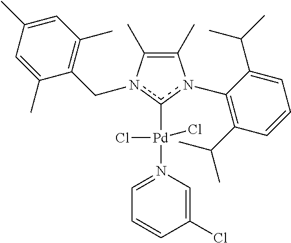 | 3-chloropyridine [1-(2,6-diisopropylphenyl)- 4,5-dimethyl-3-(2,4,6-trimethylbenzyl)-1H- imidazol-2-ylidene] dichloropalladium(II) |
| 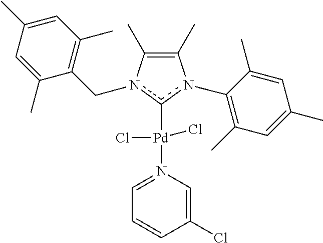 | 3-chloropyridine [1-mesityl-4,5-dimethyl-3- (2,4,6-trimethylbenzyl)-1H-imidazol-2-ylidene] dichloropalladium(II) |
| 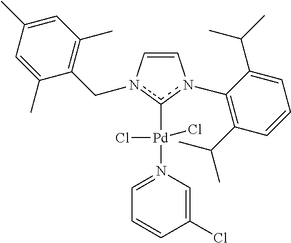 | 3-chloropyridine [1-(2,6-diisopropylphenyl)-3- (2,4,6-trimethylbenzyl)-1H-imidazol-2-ylidene] dichloropalladium(II) |
| 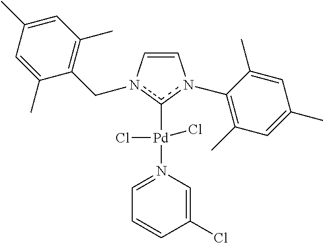 | 3-chloropyridine [1-mesityl-3-(2,4,6- trimethylbenzyl)-1H-imidazol-2-ylidene] dichloropalladium(II) |
| 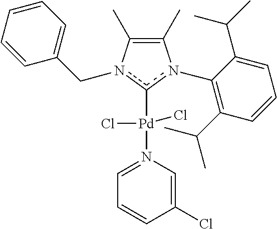 | 3-chloropyridine [1-(2,6-diisopropylphenyl)- 4,5-dimethyl-3-benzyl-1H-imidazol-2-ylidene] dichloropalladium(II) |

### Methods

In certain embodiments, the present disclosure provides a method of promoting a reaction between an alkyne and a borylation reagent, the method comprising contacting the alkyne and the borylation reagent in the presence of a base, a protic solvent or an electrophile, and a compound of the present disclosure.

In certain embodiments, the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂. In certain embodiments, the alkyne is a terminal alkyne.

In certain embodiments, the borylation reagent is a diboronic ester. In certain embodiments, the diboronic ester is B₂(pin)₂.

In certain embodiments, the compound of the present disclosure is present in an amount of about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.00, 1.10, 1.20, 1.30, 1.40, 1.50, 1.60, 1.70, 1.80, 1.90, 2.00, 20 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90, or about 5.0 mol %.

In certain embodiments, the base is selected from the group consisting of NaOH and NaOtBu.

In certain embodiments, the protic solvent is methanol. In certain embodiments, the protic solvent is ethanol. In certain embodiments, the protic solvent is isopropanol. In certain embodiments, the protic solvent is n-butanol. In certain embodiments, the protic solvent is water.

In certain embodiments, the method further comprises an aprotic solvent. In certain embodiments, the aprotic solvent is tetrahydrofuran. In certain embodiments, the aprotic solvent is 1,4-dioxane. In certain embodiments, the aprotic solvent is Et₂O. In certain embodiments, the aprotic solvent is DMF.

In certain embodiments, the electrophile is selected from the group consisting of C₁-C₁₂ haloalkyl, C₆-C₁₂ haloaralkyl, C₆-C₁₀ aryl halide, C₄-C₁₀ heteroaryl halide, C₁-C₁₂ alkyl-(C═O)Cl, C₆-C₁₀ aryl-(C═O)Cl, C₄-C₁₀ heteroaryl-(C═O)Cl, and CO₂. In certain embodiments, the electrophile is MeI.

In certain embodiments, the contacting occurs at a temperature of about 10, 20, 23, 30, 40, 50, 60, 70, 80, 90, 100, 110, 120, 130, 140, or about 150° C. In certain embodiments, the present disclosure further provides a method of promoting hydration of a first reagent, the method comprising contacting the first reagent and water in the presence of a compound of the present disclosure.

In certain embodiments, the first reagent comprises an alkyne. In certain embodiments, the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the method comprises a Lewis acid. In certain embodiments, the Lewis acid is selected from the group consisting of AgNTf₂, AgOAc, AgOTf, NaBArF, KB(C₆F₅)₄, and AgSbF₆.

In certain embodiments, the compound of the present disclosure is present in an amount of about 10, 20, 30, 40, 50, 60, 70, 80, 90, 100, 110, 120, 130, 140, 150, 160, 170, 180, 190, or about 200 ppm.

In certain embodiments, the method further comprises a solvent. In certain embodiments, solvent is 1,4-dioxane. In certain embodiments, the solvent is tetrahydrofuran. In certain embodiments, the solvent is diethyl ether. In certain embodiments, the solvent is DMF. In certain embodiments, the solvent and water have a ratio of about 2:1.

In certain embodiments, the contacting occurs at a temperature of about 10, 20, 23, 30, 40, 50, 60, 70, 80, 90, 100, 110, 120, 130, 140, or about 150° C.

In certain embodiments, the first reagent is selected from the group consisting of optionally substituted C₁-C₁₂ alkyl nitrile, optionally substituted C₂-C₆ alkenyl nitrile, optionally substituted C₆-C₁₀ aryl nitrile, optionally substituted C₄-C₁₀ heteroaryl nitrile, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the method further comprises a solvent. In certain embodiments, the solvent is tetrahydrofuran. In certain embodiments, solvent is 1,4-dioxane. In certain embodiments, the solvent is diethyl ether. In certain embodiments, the solvent is DMF. In certain embodiments, the solvent and water have a ratio of about 1:1.

In certain embodiments, the contacting occurs at a temperature of about 90, 100, 110, 120, 130, 140, 150, 160, or about 170° C.

### EXAMPLES

Various embodiments of the present application can be better understood by reference to the following Examples which are offered by way of illustration. The scope of the present application is not limited to the Examples given herein.

### Example 1

### Synthesis of Unsymmetrical NHC Ligands

Synthesis of 1-(2,6-diisopropylphenyl)-4,5-dimethyl-1H-imidazole

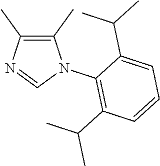 [SMILES: Cc1ncn(-c2c(C(C)C)cccc2C(C)C)c1C]

An oven-dried 250 mL round-bottomed flask equipped with a stir bar was charged with diacetyl (861 mg, 10 mmol), 2,6-diisopropylaniline (12 mmol), NH₄OAc (925 mg, 12 mmol), paraformaldehyde (480 mg, 10 mmol) and H₂O (0.5 mL). Then, CHCl₃ (20 mL) and acetic acid (3.0 g, 50 mmol) was added. The mixture was refluxed and monitored by TLC until completion (48 h). After removal of the solvent, the dark residue was dissolved in Et₂O and basified to pH 14 in an ice bath with aqueous 40% KOH solution. The resulting mixture was extracted with Et₂O (5×50 mL), and the combined organic layers were washed with H₂O, brine and dried over Na₂SO₄. The crude product was concentrated and purified by silica gel column chromatography to provide the title compound as a white solid (1.4 g, 55% yield). ¹HNMR (500 MHz, CDCl₃) δ 7.42 (t, J=7.8 Hz, 1H), 7.27 (d, J=7.8 Hz, 2H), 7.24 (s, 1H), 2.40-2.32 (m, 2H), 2.26 (s, 3H), 1.86 (s, 3H), 1.14 (d, J=7.1 Hz, 6H), 1.09 (d, J=7.1 Hz, 6H). ¹³C NMR (125 MHz, CDCl₃) δ 146.97, 135.58, 133.61, 131.78, 129.69, 123.79, 123.62, 27.96, 25.18, 23.19, 12.99, 8.45.

Synthesis of 1-mesityl-4,5-dimethyl-1H-imidazole

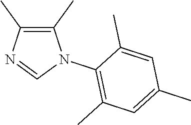

An oven-dried 250 mL round-bottomed flask equipped with a stir bar was charged with diacetyl (861 mg, 10 mmol), 2,4,6-trimethylaniline (12 mmol), NH₄OAc (925 mg, 12 mmol), paraformaldehyde (480 mg, 10 mmol) and H₂O (0.5 mL). Then, CHCl₃ (20 mL) and acetic acid (3.0 g, 50 mmol) was added. The mixture was refluxed and monitored by TLC until completion (48 h). After removal of the solvent, the dark residue was dissolved in Et₂O and basified to pH 14 in an ice bath with aqueous 40% KOH solution. The resulting mixture was extracted with Et₂O (5×50 mL), and the combined organic layers were washed with H₂O, brine and dried over Na₂SO₄. The crude product was concentrated and purified by silica gel column chromatography to provide the title compound as a white solid (1.35 g, 63% yield). ¹H NMR (500 MHz, CDCl₃) δ 7.25 (s, 1H), 6.97 (s, 2H), 2.34 (s, 3H), 2.25 (s, 3H), 1.93 (s, 6H), 1.85 (s, 3H). ¹³C NMR (125 MHz, CDCl₃) δ 138.67, 136.03, 134.40, 133.72, 132.43, 128.91, 122.60, 21.04, 17.39, 12.93, 8.15.

Synthesis of 1-(2,6-diisopropylphenyl)-1H-imidazole

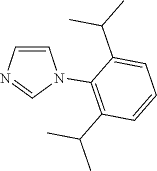 [SMILES: CC(C)c1cccc(C(C)C)c1-n1ccnc1]

An oven-dried 250 mL round-bottomed flask equipped with a stir bar was charged with diacetyl (861 mg, 10 mmol), 2,6-diisopropylaniline (10 mmol), NH₄OAc (1.54 g, 20 mmol), paraformaldehyde (960 mg, 20 mmol). Then, MeOH (40 mL) was injected and H₃PO₄ (3.0 g, 50 mmol) was added over a period of 10 min. The mixture was refluxed and monitored by TLC until completion (16 h). After removal of the solvent, the dark residue was dissolved in Et₂O and basified to pH 9 in an ice bath with aqueous 40% KOH solution. The resulting mixture was extracted with Et₂O (5×50 mL), and the combined organic layers were washed with H₂O, brine and dried over Na₂SO₄. The crude product was concentrated and purified by silica gel column chromatography to provide the title compound as a white solid (821 mg, 36% yield). ¹H NMR (500 MHz, CDCl₃) δ 7.49-7.40 (m, 2H), 7.29-7.24 (m, 3H), 6.95 (s, 1H), 2.43-2.39 (m, 2H), 1.14 (d, J=7.2 Hz, 12H). ¹³C NMR (125 MHz, CDCl₃) δ 146.51, 138.46, 132.84, 129.80, 129.35, 123.74, 121.54, 28.08, 24.42, 24.34.

Synthesis of 1-mesityl-1H-imidazole

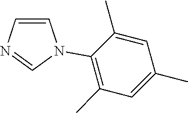

An oven-dried 250 mL round-bottomed flask equipped with a stir bar was charged with diacetyl (861 mg, 10 mmol), aniline derivative (10 mmol), NH₄OAc (1.54 g, 20 mmol), paraformaldehyde (960 mg, 20 mmol). Then, MeOH (40 mL) was injected and H₃PO₄ (3.0 g, 50 mmol) was added over a period of 10 min. The mixture was refluxed and monitored by TLC until completion (16 h). After removal of the solvent, the dark residue was dissolved in Et₂O and basified to pH 9 in an ice bath with aqueous 40% KOH solution. The resulting mixture was extracted with Et₂O (5×50 mL), and the combined organic layers were washed with H₂O, brine and dried over Na₂SO₄. The crude product was concentrated and purified by silica gel column chromatography to provide the title compound as a white solid (651 mg, 35% yield). ¹H NMR (500 MHz, CDCl₃) δ 7.42 (s, 1H), 7.22 (s, 1H), 6.96 (s, 1H), 6.88 (s, 1H), 2.33 (s, 1H), 1.98 (s, 1H). ¹³C NMR (125 MHz, CDCl₃) δ 138.81, 137.45, 135.38, 133.39, 129.52, 128.97, 120.03, 20.99, 17.29.

Synthesis of 1-(2,6-diisopropylphenyl)-4,5-dimethyl-3-(2,4,6-trimethylbenzyl)-1H-imidazol-3-ium chloride (NHC-1)

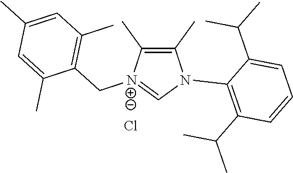 [SMILES: Cc1cc(C)c(C[n+]2cn(-c3c(C(C)C)cccc3C(C)C)c(C)c2C)c(C)c1 ; [Cl-]]

An oven-dried 10 mL tube equipped with a stir bar was charged with 1-(2,6-diisopropylphenyl)-4,5-dimethyl-1H-imidazole (2.0 mmol) and 2-(chloromethyl)-1,3,5-trimethylbenzene (2.0 mmol) in dry THF (2 mL). The mixture was refluxed for 15 h under Ar and then evaporated until THF was moderately reduced without complete drying. The resulting solids were filtered and washed with Et₂O to provide the title compound as a white solid (468 mg, 55% yield). ¹H NMR (500 MHz, CDCl₃) δ 9.35 (s, 1H), 7.50 (t, J=7.8 Hz, 1H), 7.27 (d, J=7.8 Hz, 2H), 6.86 (s, 2H), 5.91 (s, 2H), 2.99 (s, 3H), 2.28 (s, 3H), 2.24 (s, 3H), 2.18-2.13 (m, 2H), 1.91 (s, 3H), 1.16 (d, J=7.1 Hz, 6H), 1.09 (d, J=7.1 Hz, 6H). ¹³C NMR (125 MHz, CDCl₃) δ 145.66, 139.35, 137.81, 135.51, 131.92, 130.02, 128.61, 128.51, 128.21, 125.61, 124.84, 47.75, 28.67, 25.19, 23.00, 20.97, 19.99, 9.47, 8.55.

Synthesis of 1-(2,6-diisopropylphenyl)-3-(2,4,6-trimethylbenzyl)-1H-imidazol-3-ium chloride (NHC-2)

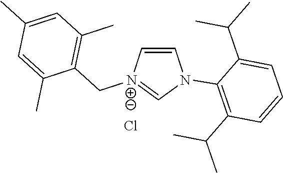 [SMILES: Cc1cc(C)c(C[n+]2ccn(-c3c(C(C)C)cccc3C(C)C)c2)c(C)c1 ; [Cl-]]

An oven-dried 10 mL tube equipped with a stir bar was charged with 1-(2,6-diisopropylphenyl)-1H-imidazole (2.0 mmol) and 2-(chloromethyl)-1,3,5-trimethylbenzene (2.0 mmol) in dry THF (2 mL). The mixture was refluxed for 15 h under Ar and then evaporated until THF was moderately reduced without complete drying. The resulting solids were filtered and washed with Et₂O to provide the title compound as a white solid (428 mg, 54% yield). ¹H NMR (500 MHz, CDCl₃) δ 10.63 (s, 1H), 7.52 (t, J=7.8 Hz, 1H), 7.41 (s, 1H), 7.29 (d, J=7.8 Hz, 2H), 7.16 (s, 1H), 6.94 (s, 2H), 6.07 (s, 2H), 2.35 (s, 6H), 2.30 (s, 3H), 2.28-2.25 (m, 2H), 1.23 (d, J=7.1 Hz, 6H), 1.15 (d, J=7.1 Hz, 6H). ¹³C NMR (125 MHz, CDCl₃) δ 145.31, 139.79, 138.69, 138.14, 131.90, 130.23, 129.97, 126.14, 124.68, 124.14, 121.79, 48.72, 28.78, 24.43, 24.06, 21.06, 19.77.

Synthesis of 1-mesityl-4,5-dimethyl-3-(2,4,6-trimethylbenzyl)-1H-imidazol-3-ium chloride (NHC-3)

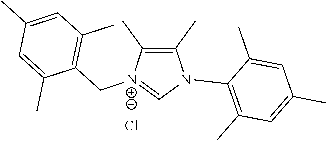

An oven-dried 10 mL tube equipped with a stir bar was charged with 1-mesityl-4,5-dimethyl-1H-imidazole (2.0 mmol) and 2-(chloromethyl)-1,3,5-trimethylbenzene (2.0 mmol) in dry THF (2 mL). The mixture was refluxed for 15 h under Ar and then evaporated until THF was moderately reduced without complete drying. The resulting solids were filtered and washed with Et₂O to provide the title compound as a white solid (506 mg, 66% yield). ¹H NMR (500 MHz, CDCl₃) δ 9.80 (s, 1H), 7.00 (s, 2H), 6.88 (s, 2H), 5.96 (s, 2H), 2.33 (s, 9H), 2.27 (s, 3H), 2.14 (s, 3H), 1.99 (s, 6H), 1.92 (s, 6H). ¹³C NMR (125 MHz, CDCl₃) δ 141.30, 139.10, 137.73, 136.23, 134.75, 130.03, 129.93, 128.97, 128.21, 127.46, 126.04, 47.82, 21.10, 20.97, 20.26, 17.64, 9.27, 8.22.

Synthesis of 1-mesityl-3-(2,4,6-trimethylbenzyl)-1H-imidazol-3-ium chloride (NHC-4)

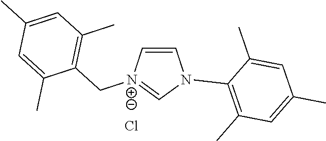

An oven-dried 10 mL tube equipped with a stir bar was charged with 1-mesityl-1H-imidazole (2.0 mmol) and 2-(chloromethyl)-1,3,5-trimethylbenzene (2.0 mmol) in dry THF (2 mL). The mixture was refluxed for 15 h under Ar and then evaporated until THF was moderately reduced without complete drying. The resulting solids were filtered and washed with Et₂O to provide the title compound as a white solid (391 mg, 55% yield). ¹H NMR (500 MHz, CDCl₃) δ 10.96 (s, 1H), 7.14 (s, 1H), 7.08 (s, 1H), 7.02 (s, 2H), 6.96 (s, 2H), 6.04 (s, 2H), 2.36 (s, 9H), 2.32 (s, 3H), 2.09 (s, 6H). ¹³C NMR (125 MHz, CDCl₃) δ 141.38, 139.76, 138.94, 138.21, 134.16, 130.77, 129.95, 129.93, 126.06, 122.84, 121.21, 48.60, 21.10, 21.07, 19.90, 17.59.

Synthesis of 1-(2,6-diisopropylphenyl)-4,5-dimethyl-3-benzyl-1H-imidazol-3-ium chloride (NHC-5a)

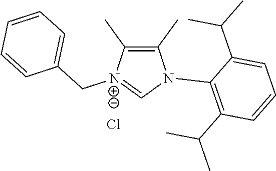 [SMILES: Cc1c(C)[n+](Cc2ccccc2)cn1-c1c(C(C)C)cccc1C(C)C ; [Cl-]]

An oven-dried 10 mL tube equipped with a stir bar was charged with 1-(2,6-diisopropylphenyl)-4,5-dimethyl-1H-imidazole (2.0 mmol) and benzyl chloride (2.0 mmol) in dry THF (2 mL). The mixture was refluxed for 15 h under Ar and then evaporated until THF was moderately reduced without complete drying. The resulting solids were filtered and washed with Et₂O to provide the title compound as a white solid (421 mg, 55% yield). ¹H NMR (500 MHz, CDCl₃) δ 10.91 (s, 1H), 7.56 (t, J=7.8 Hz, 1H), 7.45-7.33 (m, 7H), 6.03 (s, 2H), 2.29 (s, 3H), 2.25-2.20 (m, 2H), 1.93 (s, 3H), 1.27 (d, J=7.1 Hz, 6H), 1.19 (d, J=7.1 Hz, 6H). ¹³C NMR (125 MHz, CDCl₃) δ 145.74, 138.03, 134.06, 131.86, 129.27, 128.77, 128.41, 128.05, 127.87, 126.88, 124.84, 51.13, 28.82, 25.24, 23.11, 9.23, 8.54.

### Example 2

### Gram-Scale Chromatography-Free Ligand Synthesis

Synthesis of 1-(2,6-diisopropylphenyl)-4,5-dimethyl-3-(2,4,6-trimethylbenzyl)-1H-imidazol-3-ium chloride (Gram Scale)

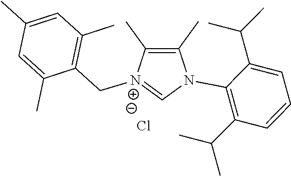 [SMILES: Cc1cc(C)c(C[n+]2cn(-c3c(C(C)C)cccc3C(C)C)c(C)c2C)c(C)c1 ; [Cl-]]

An oven-dried 250 mL round-bottomed flask equipped with a stir bar was charged with diacetyl (861 mg, 10.0 mmol, 1.0 equiv), 2,6-diisopropylaniline (1.78 g, 10.0 mmol, 1.0 equiv), ammonium acetate (848 mg, 11.0 mmol, 1.1 equiv), paraformaldehyde (300 mg, 10.0 mmol, 1.0 equiv) and water (0.5 mL). Then CHCl₃ (20 mL) and acetic acid (3.0 g, 50.0 mmol, 5.0 equiv) were added, and the reaction mixture was refluxed for 48 h. After the indicated time, the solvent was removed under reduced pressure. The dark residue was diluted with water (50 mL) and washed with EtOAc (50 mL). The EtOAc layer was re-extracted with aqueous 2 N HCl (3×15 mL), and the aqueous layer was collected and organic layer was discarded. The combined aqueous layer was basified (pH>10) with aqueous 25% NaOH, and extracted with EtOAc (3×25 mL). The combined organic layer was dried, filtered through 2 inch thick silica and concentrated. To the crude intermediate was added 2-(chloromethyl)-1,3,5-trimethylbenzene (1.27 g, 7.5 mmol, 0.75 equiv) and dry THF (10 mL), and the mixture was degassed with argon for 1 min and sealed. The reaction mixture was then stirred at 70° C. for 15 h. After the indicated time, the solvent was removed under reduced pressure. The resulting solid was washed with diethyl ether (20 mL) and EtOAc (20 mL) and dried to obtain pure product. White solid. Yield 42.3% (1.80 g). Mp=257-258° C. ¹H NMR (500 MHz, CDCl₃) δ 9.45 (s, 1H), 7.48 (t, J=7.8 Hz, 1H), 7.30-7.20 (m, 2H), 6.83 (s, 2H), 5.92 (s, 2H), 3.94-3.88 (m, 1H), 2.27 (s, 6H), 2.24 (s, 3H), 2.21 (s, 3H), 2.16 -2.10 (m, 2H), 1.88 (s, 3H), 1.11 (dd, J=26.4, 6.8 Hz, 12H). ¹³C NMR (126 MHz, CDCl₃) δ 145.62, 139.25, 137.75, 135.69, 131.85, 129.96, 128.50, 128.39, 128.18, 125.64, 124.79, 47.76, 28.63, 25.16, 22.96, 20.92, 19.98, 9.42, 8.49. HRMS (ESI) m/z: [2M-Cl]⁺ Calcd for C₅₄H₇₄N₄Cl 813.5597; Found 813.5591.

Synthesis of 1-(2,6-diisopropylphenyl)-4,5-dimethyl-3-(2,4,6-trimethylbenzyl)-1H-imidazol-3-ium chloride (Decagram Scale)

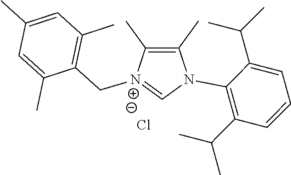 [SMILES: Cc1cc(C)c(C[n+]2cn(-c3c(C(C)C)cccc3C(C)C)c(C)c2C)c(C)c1 ; [Cl-]]

An oven-dried 250 mL round-bottomed flask equipped with a stir bar was charged with diacetyl (8.61 g, 100 mmol, 1.0 equiv), 2,6-diisopropylaniline (17.8 g, 100 mmol, 1.0 equiv), ammonium acetate (8.48 g, 11 mmol, 1.1 equiv), paraformaldehyde (3.0 g, 100 mmol, 1.0 equiv) and water (5.0 mL). Then CHCl₃ (150 mL) and acetic acid (30.0 g, 500 mmol, 5.0 equiv) was added, and the reaction mixture was refluxed for 72 h. After the indicated time, the solvent was removed under reduced pressure. The dark residue was diluted with water (100 mL) and washed with EtOAc (150 mL). The EtOAc layer was re-extracted with aqueous 2 N HCl (3×50 mL), and the aqueous layer was collected and organic layer was discard. The combined aqueous layer was basified (pH>10) with aqueous 25% NaOH, and extracted with EtOAc (3×75 mL). The combined organic layer was dried, filtered through 3˜4 inch thick silica and concentrated. To the crude intermediate was added 2-(chloromethyl)-1,3,5-trimethylbenzene (12.7 g, 75 mmol, 0.75 equiv) and dry THF (75 mL), and the mixture was degassed with argon for 2 min and sealed. The reaction mixture was then stirred at 70° C. for 15 h. After the indicated time, the solvent was removed under reduced pressure. The resulting solid was washed with diethyl ether (50 mL) and EtOAc (50 mL) and dried to obtain pure product. White solid. Yield 36.5% (15.5 g). Mp=257-258° C. ¹H NMR (500 MHz, CDCl₃) δ 9.45 (s, 1H), 7.48 (t, J=7.8 Hz, 1H), 7.30-7.20 (m, 2H), 6.83 (s, 2H), 5.92 (s, 2H), 3.94-3.88 (m, 1H), 2.27 (s, 6H), 2.24 (s, 3H), 2.21 (s, 3H), 2.16-2.10 (m, 2H), 1.88 (s, 3H), 1.11 (dd, J=26.4, 6.8 Hz, 12H). ¹³C NMR (126 MHz, CDCl₃) δ 145.62, 139.25, 137.75, 135.69, 131.85, 129.96, 128.50, 128.39, 128.18, 125.64, 124.79, 47.76, 28.63, 25.16, 22.96, 20.92, 19.98, 9.42, 8.49. HRMS (ESI) m/z: [2M-Cl]⁺ Calcd for C₅₄H₇₄N₄Cl 813.5597; Found 813.5591.

Synthesis of 1-mesityl-4,5-dimethyl-3-(2,4,6-trimethylbenzyl)-1H-imidazol-3-ium chloride (Gram Scale)

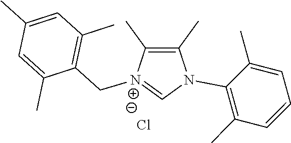 [SMILES: Cc1cc(C)c(C[n+]2cn(-c3c(C)cccc3C)c(C)c2C)c(C)c1 ; [Cl-]]

An oven-dried 250 mL round-bottomed flask equipped with a stir bar was charged with diacetyl (861 mg, 10.0 mmol, 1.0 equiv), 2,4,6-trimethylaniline (1.35 g, 10.0 mmol, 1.0 equiv), ammonium acetate (848 mg, 11.0 mmol, 1.1 equiv), paraformaldehyde (300 mg, 10.0 mmol, 1.0 equiv) and water (0.5 mL). Then CHCl₃ (20 mL) and acetic acid (3.0 g, 50.0 mmol, 5.0 equiv) was added, and the reaction mixture was refluxed for 48 h. After the indicated time, the solvent was removed under reduced pressure. The dark residue was diluted with water (50 mL) and washed with EtOAc (50 mL). The EtOAc layer was re-extracted with aqueous 2 N HCl (3×15 mL), and the aqueous layer was collected and organic layer was discard. The combined aqueous layer was basified (pH>10) with aqueous 25% NaOH, and extracted with EtOAc (3×25 mL). The combined organic layer was dried, filtered through 2 inch thick silica and concentrated. To the crude intermediate (no need further purification), 2-(chloromethyl)-1,3,5-trimethylbenzene (1.27 g, 7.5 mmol, 0.75 equiv) and dry THF (10 mL) was added, and the mixture was degassed with argon for 1 min and sealed. The reaction mixture was then stirred at 70° C. for 15 h. After the indicated time, the solvent was removed under reduced pressure. The resulting solid was washed with diethyl ether (20 mL) and EtOAc (20 mL) and dried to obtain pure product. White solid. Yield 41.8% (1.60 g). Mp=291-292° C. ¹H NMR (500 MHz, CDCl₃) δ 9.61 (s, 1H), 6.94 (s, 2H), 6.82 (s, 2H), 5.86 (s, 2H), 2.27 (s, 3H), 2.26 (s, 6H), 2.21 (s, 3H), 2.10 (s, 3H), 1.92 (s, 6H), 1.86 (s, 3H). ¹³C NMR (126 MHz, CDCl₃) δ 141.21, 139.07, 137.60, 135.78, 134.61, 129.95, 129.83, 128.84, 128.21, 127.55, 125.79, 47.63, 21.00, 20.87, 20.12, 17.52, 9.20, 8.15. HRMS (ESI) m/z: [2M-Cl]⁺ Calcd for C₄₈H₆₂N₄Cl 729.4658; Found 729.4654.

Synthesis of 1-(2,6-diisopropylphenyl)-4,5-dimethyl-3-benzyl-1H-imidazol-3-ium chloride (Gram Scale)

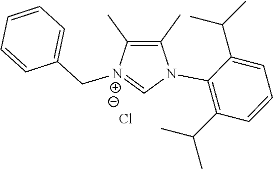 [SMILES: Cc1c(C)[n+](Cc2ccccc2)cn1-c1c(C(C)C)cccc1C(C)C ; [Cl-]]

An oven-dried 250 mL round-bottomed flask equipped with a stir bar was charged with diacetyl (861 mg, 10.0 mmol, 1.0 equiv), 2,6-diisopropylaniline (1.78 g, 10.0 mmol, 1.0 equiv), ammonium acetate (848 mg, 11.0 mmol, 1.1 equiv), paraformaldehyde (300 mg, 10.0 mmol, 1.0 equiv) and water (0.5 mL). Then CHCl₃ (20 mL) and acetic acid (3.0 g, 50.0 mmol, 5.0 equiv) was added, and the reaction mixture was refluxed for 48 h. After the indicated time, the solvent was removed under reduced pressure. The dark residue was diluted with water (50 mL) and washed with EtOAc (50 mL). The EtOAc layer was re-extracted with aqueous 2 N HCl (3×15 mL), and the aqueous layer was collected and organic layer was discard. The combined aqueous layer was basified (pH>10) with aqueous 25% NaOH, and extracted with EtOAc (3×25 mL). The combined organic layer was dried, filtered through 2 inch thick silica and concentrated. To the crude intermediate (no need further purification), benzyl chloride (950 mg, 7.5 mmol, 0.75 equiv) and dry THF (7.5 mL) was added, and the mixture was degassed with argon for 1 min and sealed. The reaction mixture was then stirred at 70° C. for 15 h. After the indicated time, the solvent was removed under reduced pressure. The resulting solid was washed with diethyl ether (20 mL) and EtOAc (20 mL) and dried to obtain pure product. White solid. Yield 31.3% (1.20 g). Mp=195-196° C. ¹H NMR (500 MHz, CDCl₃) δ 10.79 (s, 1H), 7.52 (t, J=7.8 Hz, 1H), 7.41 -7.27 (m, 7H), 5.98 (s, 2H), 2.26 (s, 3H), 2.21-2.15 (m, 2H), 1.89 (s, 3H), 1.19 (dd, J=36.2, 6.8 Hz, 12H). ¹³C NMR (126 MHz, CDCl₃) δ 145.72, 137.77, 134.02, 131.84, 129.24, 128.74, 128.41, 128.07, 127.91, 127.06, 124.81, 51.06, 28.79, 25.25, 23.08, 9.27, 8.54. HRMS (ESI) m/z: [2M-Cl]⁺ Calcd for C₄₈H₆₂N₄Cl 729.4658; Found 729.4664.

Synthesis of 1-(2,6-diisopropylphenyl)-4,5-dimethyl-3-benzyl-1H-imidazol-3-ium bromide (Gram Scale)

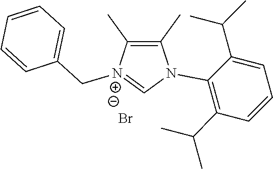 [SMILES: Cc1c(C)[n+](Cc2ccccc2)cn1-c1c(C(C)C)cccc1C(C)C ; [Br-]]

An oven-dried 250 mL round-bottomed flask equipped with a stir bar was charged with diacetyl (861 mg, 10.0 mmol, 1.0 equiv), 2,6-diisopropylaniline (1.78 g, 10.0 mmol, 1.0 equiv), ammonium acetate (848 mg, 11.0 mmol, 1.1 equiv), paraformaldehyde (300 mg, 10.0 mmol, 1.0 equiv) and water (0.5 mL). Then CHCl₃ (20 mL) and acetic acid (3.0 g, 50.0 mmol, 5.0 equiv) was added, and the reaction mixture was refluxed for 48 h. After the indicated time, the solvent was removed under reduced pressure. The dark residue was diluted with water (50 mL) and washed with EtOAc (50 mL). The EtOAc layer was re-extracted with aqueous 2 N HCl (3×15 mL), and the aqueous layer was collected and organic layer was discard. The combined aqueous layer was basified (pH>10) with aqueous 25% NaOH, and extracted with EtOAc (3×25 mL). The combined organic layer was dried, filtered through 2 inch thick silica and concentrated. To the crude intermediate (no need further purification), benzyl bromide (1.28 g, 7.5 mmol, 0.75 equiv) and dry THF (7.5 mL) was added, and the mixture was degassed with argon for 1 min and sealed. The reaction mixture was then stirred at 70° C. for 30 min. After the indicated time, diethyl ether (20 mL) was added. The resulting precipitation was filtered and dried to obtain pure product. White solid. Yield 39.8% (1.70 g). Mp=236-237° C. ¹H NMR (500 MHz, CDCl₃) δ 10.56 (s, 1H), 7.53 (t, J=7.8 Hz, 1H), 7.41-7.30 (m, 7H), 6.00 (s, 2H), 2.28 (s, 3H), 2.22-2.16 (m, 2H), 1.90 (s, 3H), 1.20 (dd, J=35.0, 6.8 Hz, 12H). ¹³C NMR (126 MHz, CDCl₃) δ 145.74, 136.94, 133.78, 131.94, 129.29, 128.85, 128.28, 128.08, 128.06, 127.35, 124.87, 51.07, 28.80, 25.32, 23.10, 9.37, 8.55. HRMS (ESI) m/z: [2M-Br]⁺ Calcd for C₄₈H₆₂N₄Br 775.4135; Found 775.4136.

Synthesis of 1-(2,6-diisopropylphenyl)-3-(2,4,6-trimethylbenzyl)-1H-imidazol-3-ium chloride (Gram Scale)

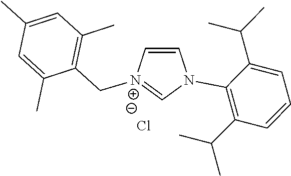 [SMILES: Cc1cc(C)c(C[n+]2ccn(-c3c(C(C)C)cccc3C(C)C)c2)c(C)c1 ; [Cl-]]

An oven-dried 250 mL round-bottomed flask equipped with a stir bar was charged with glyoxal (40% wt. in water, 1.60 g, 11.0 mmol, 1.1 equiv), formaldehyde (37% wt. in water, 892 mg, 11.0 mmol, 1.1 equiv) and AcOH (3.0 ml), and stirred at room temperature for 30 min. In a separate conical flask was added 2,6-diisopropylaniline (1.77 g, 10.0 mmol, 1.0 equiv), ammonium acetate (848 mg, 11.0 mmol, 1.1 equiv), AcOH (3.0 mL) and water (0.3 mL), and the mixture was stirred at room temperature for 30 min to form a viscous solution. Then the viscous solution was added to the round-bottomed flask slowly over 5 min. The reaction mixture was then stirred at 80° C. for 15 h. After the indicated time, the solvent was removed under reduced pressure. The dark residue was diluted with water (50 mL) and washed with EtOAc (50 mL). The EtOAc layer was re-extracted with aqueous 2 N HCl (3×15 mL), and the aqueous layer was collected and organic layer was discard. The combined aqueous layer was basified (pH>10) with aqueous 25% NaOH, and extracted with EtOAc (3×25 mL). The combined organic layer was dried, filtered through 2 inch thick silica and concentrated. To the crude intermediate (no need further purification), 2-(chloromethyl)-1,3,5-trimethylbenzene (1.27 g, 7.5 mmol, 0.75 equiv) and dry THF (10 mL) was added, and the mixture was degassed with argon for 1 min and sealed. The reaction mixture was then stirred at 70° C. for 15 h. After the indicated time, the solvent was removed under reduced pressure. The resulting solid was washed with diethyl ether (20 mL) and EtOAc (20 mL) and dried to obtain pure product. White solid. Yield 21.7% (0.86 g). Mp=256-257° C. ¹H NMR (500 MHz, CDCl₃) δ 10.66 (s, 1H), 7.50 (t, J=7.8 Hz, 1H), 7.38 (s, 1H), 7.32-7.21 (m, 2H), 7.14 (s, 1H), 6.92 (s, 2H), 6.06 (s, 2H), 2.33 (s, 6H), 2.29-2.21 (m, 5H), 1.17 (dd, J=42.9, 6.7 Hz, 12H). ¹³C NMR (126 MHz, CDCl₃) δ 145.28, 139.76, 138.72, 138.11, 131.88, 130.23, 129.96, 126.14, 124.66, 124.13, 121.73, 48.67, 28.77, 24.44, 24.05, 21.05, 19.78. HRMS (ESI) m/z: [2M-Cl]⁺ Calcd for C₅₀H₆₆N₄Cl 757.4971; Found 757.4964.

### Example 3

### Electronic Parameters of NHCs

### Tolman Electronic Parameter (TEP)

The NHC ligands of the present disclosure were characterized by the Tolman electronic parameter (TEP) using the CO stretching frequencies of the corresponding [Ir(NHC)(CO)₂Cl] complexes prepared from NHC ligands: NHC-1 (2049.5 cm⁻¹); NHC-2 (2081.8 cm⁻¹); NHC-3 (2048.9 cm⁻¹); NHC-4 (2082.9 cm⁻¹); and NHC-5a (2051.2 cm⁻¹) vs IMes=2051 cm⁻¹/IPr=2052 cm⁻¹.

### ⁷⁷Se-NMR Studies of Selenium Adducts

The NHC ligands of the present disclosure were characterized by ⁷⁷Se-NMR using Selenium adducts [Se(NHC)] prepared from NHC ligands: NHC-1 (61.43 ppm); NHC-2 (38.77 ppm); NHC-3 (25.90 ppm); NHC-4 (13.50 ppm); and NHC-5a (43.62 ppm) vs IMes (35 ppm) and IPr (87 ppm).

### Example 4

### Synthesis of Unsymmetrical NHC Catalyst Complexes

### General Procedure A: Synthesis of Cu(I)-NHC Complexes

An oven-dried 10 mL vial equipped with a stir bar was charged with imidazolium chloride (0.2 mmol, 1.0 equiv), NaOtBu (0.2 mmol, 1.0 equiv), and CuCl (20 mg, 0.2 mmol, 1.0 equiv) in dry THF (5 ml). The reaction was fully bubbled with Argon and stirred at room temperature overnight. The mixture was diluted with DCM and filtered out (eluting with DCM). The solution was collected and concentrated by evaporation. The pure product was obtained by participation from hexane as a solid.

### General Procedure B: Synthesis of Au(I)-NHC Complexes

An oven-dried 10 mL vial equipped with a stir bar was charged with imidazolium chloride (0.2 mmol, 1.0 equiv), K₂CO₃ (0.24 mmol, 1.2 equiv), and Au(SMe₂)Cl (59 mg, 0.2 mmol, 1.0 equiv) in acetone (5 ml). The reaction was fully bubbled with Argon and stirred at 60° C. overnight. The mixture was diluted with DCM and filtered out (eluting with DCM). The solution was collected and concentrated by evaporation. The pure product was obtained by participation from hexane as a solid.

### General Procedure C: Synthesis of Ag(I)-NHC Complexes

An oven-dried 10 mL vial equipped with a stir bar was charged with imidazolium chloride (0.2 mmol, 1.0 equiv) and Ag₂O (24 mg, 0.1 mmol, 0.5 equiv) in DCM (5 ml). The reaction was fully bubbled with Argon and stirred at room temperature without light overnight. The mixture was diluted with DCM and filtered out (eluting with DCM). The solution was collected and concentrated by evaporation. The pure product was obtained by participation from hexane as a solid.

### General Procedure D: Synthesis of Pd(II)-NHC (Optionally Substituted Allyl) Complexes

An oven-dried 10 mL vial equipped with a stir bar was charged with imidazolium chloride (0.1 mmol, 1.0 equiv), [Pd(cinnamyl)Cl]₂ (25.9 mg, 0.05 mmol, 0.5 equiv) and K₂CO₃ (14 mg, 0.1 mmol, 1.0 equiv) in acetone (2 ml). The reaction was stirred at 60° C. for 16 h. The mixture was diluted with DCM and filtered through a pad of silica. The solution was collected and concentrated by evaporation. The pure product was obtained by precipitation with hexane as a yellow solid.

### General Procedure E: Synthesis of Pd(II)-NHC (Pyridine-Enhanced Precatalyst Preparation Stabilization (PEPPSI)) Complexes

An oven-dried 10 mL vial equipped with a stir bar was charged with imidazolium chloride (0.05 mmol, 1.0 equiv), PdCl₂ (9.8 mg, 0.055 mmol, 1.1 equiv) and K₂CO₃ (35 mg, 0.25 mmol, 5.0 equiv) in 3-chloropyridine (2 ml). The reaction was stirred at 80° C. for 16 h. The mixture was diluted with DCM and filtered through a pad of silica. The solution was collected and concentrated by evaporation. The pure product was obtained by precipitation with hexane as a yellow solid.

Synthesis of 1-(2,6-diisopropylphenyl)-4,5-dimethyl-3-(2,4,6-trimethylbenzyl)-imidazol-2-ylidene copper(I) chloride [Cu(NHC-1)Cl]

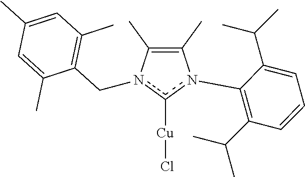

Following General Procedure A using NHC-1 the title compound was obtained as a white solid (70 mg, 72% yield). ¹H NMR (500 MHz, CDCl₃) δ 7.44 (t, J=7.8 Hz, 1H), 7.24 (d, J=7.8 Hz, 2H), 5.40 (s, 2H), 5.91 (s, 2H), 2.38-2.33 (m, 2H), 2.31 (s, 9H), 2.01 (s, 3H), 1.79 (s, 3H), 1.24 (d, J=7.1 Hz, 6H), 1.15 (d, J=7.1 Hz, 6H). ¹³C NMR (125 MHz, CDCl₃) δ 145.90, 138.62, 137.22, 133.20, 130.20, 130.10, 127.96, 126.70, 125.02, 124.14, 48.75, 28.28, 25.41, 23.22, 21.05, 20.56, 9.49, 9.19. HRMS calcd for C₂₇H₃₆CuCl₂N₂Na (M⁺+Na) 509.1755, found 509.1772.

Synthesis of 1-(2,6-diisopropylphenyl)-3-(2,4,6-trimethylbenzyl)-imidazol-2-ylidene copper(I) chloride [Cu(NHC-2)Cl]

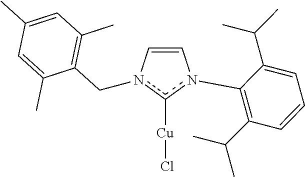

Following General Procedure A using NHC-2 the title compound was obtained as a white solid (61 mg, 66% yield). ¹H NMR (500 MHz, CDCl₃) δ 7.37 (t, J=7.8 Hz, 1H), 7.17 (d, J=7.8 Hz, 2H), 6.89 (s, 2H), 6.76 (s, 1H), 6.61 (s, 1H), 5.43 (s, 2H), 2.36-2.29 (m, 2H), 2.28 (d, J=7.1 Hz, 6H), 2.24 (s, 3H), 1.20 (d, J=7.1 Hz, 6H), 1.04 (d, J=7.1 Hz, 6H). ¹³C NMR (125 MHz, CDCl₃) δ 145.69, 139.03, 137.84, 134.61, 130.38, 129.82, 128.07, 124.13, 123.44, 119.17, 49.43, 28.37, 24.73, 24.11, 21.06, 20.01. HRMS calcd for C₂₅H₃₂CuClN₂Na (M⁺+Na) 481.1442, found 481.1458.

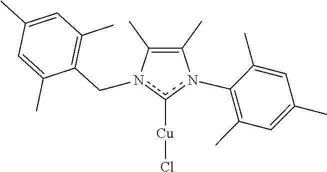

Following General Procedure A using NHC-3 the title compound was obtained as a white solid (67 mg, 75% yield). ¹H NMR (500 MHz, CDCl₃) δ 6.94 (s, 2H), 6.92 (s, 2H), 5.33 (s, 2H), 2.32 (s, 6H), 2.30 (s, 6H), 2.04 (s, 3H), 1.95 (s, 3H), 1.78 (s, 6H). ¹³C NMR (125 MHz, CDCl₃) δ 139.00, 138.60, 137.17, 134.90, 130.11, 129.38, 128.12, 48.25, 21.12, 21.05, 20.59, 17.93, 9.46, 8.83. HRMS calcd for C₂₄H₃₀CuClN₂Na (M⁺+Na) 467.1286, found 467.1298.

Synthesis of 1-mesityl-3-(2,4,6-trimethylbenzyl)-imidazol-2-ylidene copper(I) chloride [Cu(NHC-4)Cl]

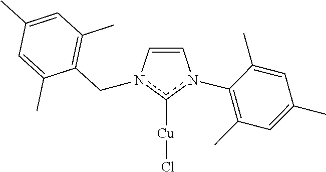

Following General Procedure A using NHC-4 the title compound was obtained as a white solid (57 mg, 68% yield). ¹H NMR (500 MHz, CDCl₃) δ 6.95 (s, 2H), 6.94 (s, 2H), 6.79 (s, 1H), 6.67 (s, 1H), 5.46 (s, 2H), 2.33 (s, 6H), 2.32 (s, 3H), 2.31 (s, 3H), 2.01 (s, 6H). ¹³C NMR (125 MHz, CDCl₃) δ 139.37, 139.00, 137.84, 135.29, 134.61, 129.79, 129.43, 128.00, 49.38, 21.08, 21.05, 20.05, 17.82. HRMS calcd for C₂₂H₂₆CuClN₂Na (M⁺+Na) 439.0973, found 439.0986.

Synthesis of 1-benzyl-3-(2,6-diisopropylphenyl)-4,5-dimethyl-imidazol-2-ylidene copper(I) chloride [Cu(NHC-5)Cl]

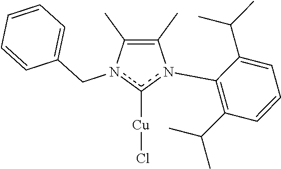

Following General Procedure A using NHC-5a the title compound was obtained as a white solid (65 mg, 73% yield). ¹H NMR (500 MHz, CDCl₃) δ 7.40-7.12 (m, 8H), 5.35 (s, 2H), 2.30-2.26 (m, 2H), 2.00 (s, 3H), 1.71 (s, 3H), 1.23 (d, J=7.1 Hz, 6H), 1.09 (d, J=7.1 Hz, 6H). ¹³C NMR (125 MHz, CDCl₃) δ 145.99, 136.00, 132.85, 130.37, 129.02, 128.19, 126.75, 124.28, 52.99, 28.38, 25.48, 23.25, 9.50, 9.27. HRMS calcd for C₂₄H₃₀CuClN₂Na (M⁺+Na) 467.1286, found 467.1292.

Synthesis of 1-(2,6-diisopropylphenyl)-4,5-dimethyl-3-(2,4,6-trimethylbenzyl)-imidazol-2-ylidene gold(I) chloride [Au(NHC-1)Cl]

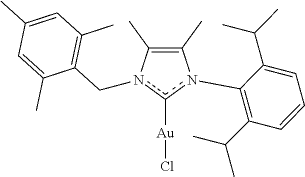

Following General Procedure B using NHC-1 the title compound was obtained as a white solid (109 mg, 88% yield). ¹H NMR (500 MHz, CDCl₃) δ 7.49 (t, J=7.8 Hz, 1H), 7.27 (d, J=7.8 Hz, 2H), 6.89 (s, 2H), 5.73 (s, 2H), 2.39-2.31 (m, 11H), 1.80 (s, 3H), 1.78 (s, 3H), 1.33 (d, J=7.1 Hz, 6H), 1.15 (d, J=7.1 Hz, 6H). ¹³C NMR (125 MHz, CDCl₃) δ 172.11, 145.97, 138.10, 137.24, 132.46, 130.47, 129.95, 128.02, 126.77, 125.30, 124.32, 50.18, 28.35, 25.17, 23.42, 20.93, 20.89, 9.51, 9.30. HRMS calcd for C₂₇H₃₆AuClN₂Na (M⁺+Na) 643.2125, found 643.2122.

Synthesis of 1-(2,6-diisopropylphenyl)-4,5-dimethyl-3-(2,4,6-trimethylbenzyl)-imidazol-2-ylidene gold(I) bistriflimide [Au(NHC-1)NTf₂]

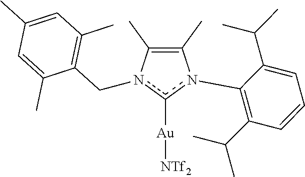

An oven-dried vial equipped with a stir bar was charged with [Au(NHC-1)Cl] (0.25 mmol, 1.0 equiv) and AgNTf₂ (0.25 mmol, 1.0 equiv, 97 mg) in 5 mL of DCM. The mixture was diluted with DCM and filtered out (eluting with DCM). The solution was collected and concentrated by evaporation. The pure product was obtained by participation from hexane as a white solid (141 mg, 65% yield). ¹H NMR (500 MHz, CDCl₃) δ 7.44 (t, J=7.2 Hz, 1H), 7.23 (d, J=7.2 Hz, 2H), 6.98 (s, 2H), 5.17 (s, 2H), 2.32 (s, 3H), 2.25 (s, 3H), 2.24 (s, 6H), 2.22-2.17 (m, 2H), 1.89 (s, 3H), 1.13 (d, J=7.2 Hz, 6H), 1.11 (d, J=7.2 Hz, 6H). ¹³C NMR (125 MHz, CDCl₃) δ 145.83, 139.40, 137.27, 133.64, 130.26, 130.23, 127.60, 127.05, 125.94, 124.27, 120.66, 118.09, 47.49, 28.26, 24.64, 23.54, 20.96, 20.00, 9.57, 9.45. ¹⁹F NMR (470 MHz, CDCl₃) δ −75.95 (s, 6F). HRMS calcd for C₂₉H₃₆AuF₆N₃O₄S₂Na (M⁺+Na) 888.1609, found 888.1617.

Synthesis of 1-(2,6-diisopropylphenyl)-3-(2,4,6-trimethylbenzyl)-imidazol-2-ylidene gold(I) chloride [Au(NHC-2)Cl]

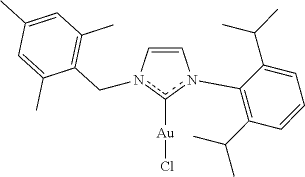

Following General Procedure B using NHC-2 the title compound was obtained as a white solid (97 mg, 82% yield). ¹H NMR (500 MHz, CDCl₃) δ 7.49 (t, J=7.8 Hz, 1H), 7.28 (d, J=7.8 Hz, 2H), 6.99 (s, 2H), 6.85 (d, J=1.8 Hz, 1H), 6.69 (d, J=1.8 Hz, 1H), 5.58 (s, 2H), 2.44-2.40 (m, 2H), 2.37 (s, 6H), 2.35 (s, 3H), 1.34 (d, J=7.1 Hz, 6H), 1.13 (d, J=7.1 Hz, 6H). ¹³C NMR (125 MHz, CDCl₃) δ 173.34, 145.72, 139.13, 137.98, 134.14, 130.59, 129.79, 127.63, 124.22, 123.31, 118.89, 49.40, 28.46, 24.44, 24.24, 21.05, 19.99. HRMS calcd for C₂₅H₃₂AuClN₂Na (M⁺+Na) 615.1812, found 615.1825.

Synthesis of 1-mesityl-4,5-dimethyl-3-(2,4,6-trimethylbenzyl)-imidazol-2-ylidene gold(I) chloride [Au(NHC-3)Cl]

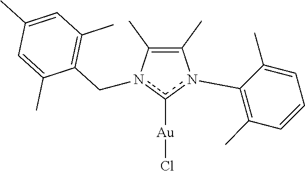

Following General Procedure B using NHC-3 the title compound was obtained as a white solid (98 mg, 85% yield). ¹H NMR (500 MHz, CDCl₃) δ 6.97 (s, 2H), 6.88 (s, 2H), 5.67 (s, 2H), 2.34 (s, 3H), 2.31 (s, 9H), 1.99 (s, 6H), 1.79 (s, 3H), 1.78 (s, 3H). ¹³C NMR (125 MHz, CDCl₃) δ 171.09, 139.41, 138.09, 137.21, 135.02, 133.27, 129.92, 129.51, 128.10, 125.72, 125.47, 49.92, 21.15, 20.96, 20.92, 17.98, 9.47, 8.90. HRMS calcd for C₂₄H₃₀AuClN₂Na (M⁺+Na) 601.1655, found 601.1659.

Synthesis of 1-mesityl-3-(2,4,6-trimethylbenzyl)-imidazol-2-ylidene gold(I) chloride [Au(NHC-4)Cl]

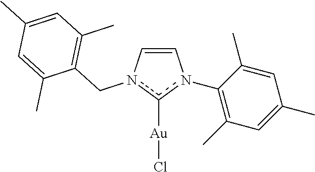

Following General Procedure B using NHC-4 the title compound was obtained as a white solid (84 mg, 76% yield). ¹H NMR (500 MHz, CDCl₃) δ 6.97 (s, 4H), 6.79 (s, 1H), 6.68 (s, 1H), 5.55 (s, 2H), 2.35 (s, 12H), 2.05 (s, 6H). ¹³C NMR (125 MHz, CDCl₃) δ 172.22, 139.55, 138.97, 137.85, 134.62, 129.61, 129.30, 127.40, 121.82, 118.99, 49.21, 29.57, 20.98, 20.90, 19.88, 17.69. HRMS calcd for C22H26AuClN2Na (M⁺+Na) 573.1342, found 573.1355.

Synthesis of 1-benzyl-3-(2,6-diisopropylphenyl)-4,5-dimethyl-imidazol-2-ylidene gold(I) chloride [Au(NHC-5)Cl]

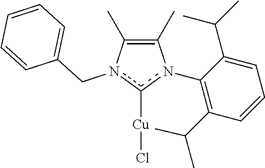

Following General Procedure B using NHC-5a the title compound was obtained as a white solid (98 mg, 85% yield). ¹H NMR (500 MHz, CDCl₃) δ 7.50 (t, J=7.8 Hz, 1H), 7.40-7.34 (m, 3H), 7.31-7.28 (m, 4H), 5.53 (s, 2H), 2.40-2.34 (m, 2H), 2.12 (s, 3H), 1.94 (s, 3H), 1.39 (d, J=7.1 Hz, 6H), 1.18 (d, J=7.1 Hz, 6H). ¹³C NMR (125 MHz, CDCl₃) δ 170.84, 146.00, 135.50, 132.34, 130.57, 129.00, 128.25, 127.21, 126.80, 124.79, 124.39, 52.65, 28.45, 25.16, 23.44, 9.65, 9.40. HRMS calcd for C₂₄H₃₀AuClN₂Na (M⁺+Na) 601.1655, found 601.1667.

Synthesis of 1-(2,6-diisopropylphenyl)-4,5-dimethyl-3-(2,4,6-trimethylbenzyl)-imidazol-2-ylidene silver(I) chloride [Ag(NHC-1)Cl]

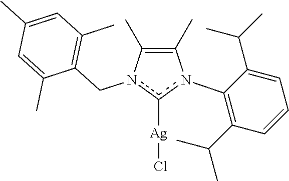

Following General Procedure C using NHC-1 the title compound was obtained as a white solid (64.8 mg, 92% yield). ¹H NMR (500 MHz, CDCl₃) δ 7.42 (t, J=7.8 Hz, 1H), 7.22 (d, J=7.8 Hz, 2H), 6.91 (s, 2H), 5.36 (s, 2H), 2.31-2.26 (m, 11H), 2.01 (s, 3H), 1.78 (s, 3H), 1.18 (d, J=7.1 Hz, 6H), 1.12 (d, J=7.1 Hz, 6H). ¹³C NMR (125 MHz, CDCl₃) δ 145.88, 138.84, 137.14, 133.46, 130.37, 130.15, 127.65, 127.27, 127.21, 125.61, 125.55, 124.31, 49.18, 28.22, 25.38, 23.33, 21.08, 20.54, 9.58, 9.37. HRMS calcd for C₂₇H₃₆AgClN₂Na (M⁺+Na) 553.1510, found 553.1525.

Synthesis of 1-(2,6-diisopropylphenyl)-3-(2,4,6-trimethylbenzyl)-imidazol-2-ylidene silver(I) chloride [Ag(NHC-2)Cl]

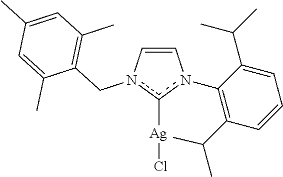

Following General Procedure C using NHC-2 the title compound was obtained as a white solid (89.7 mg, 89% yield). ¹H NMR (500 MHz, CDCl₃) δ 7.46 (t, J=7.8 Hz, 1H), 7.25 (d, J=7.8 Hz, 2H), 6.97 (s, 2H), 6.90 (s, 1H), 6.72 (s, 1H), 5.48 (s, 2H), 2.39-2.30 (m, 11H), 1.25 (d, J=7.2 Hz, 6H), 1.10 (d, J=7.2 Hz, 6H). ¹³C NMR (125 MHz, CDCl₃) δ 145.66, 139.19, 137.82, 134.75, 130.57, 129.87, 127.79, 124.27, 123.90, 123.84, 119.54, 119.48, 50.02, 29.72, 28.33, 24.64, 24.24, 21.07, 19.98. HRMS calcd for C₂₅H₃₂AgClN₂Na (M⁺+Na) 525.1197, found 525.1186.

Synthesis of 1-mesityl-4,5-dimethyl-3-(2,4,6-trimethylbenzyl)-imidazol-2-ylidene silver(I) chloride [Ag(NHC-3)Cl]

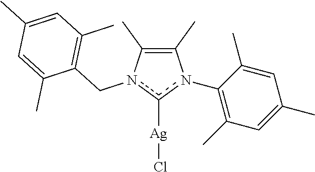

Following General Procedure C using NHC-3 the title compound was obtained as a white solid (88.2 mg, 90% yield). ¹H NMR (500 MHz, CDCl₃) δ 6.91 (s, 4H), 5.29 (s, 2H), 2.30 (s, 3H), 2.29 (s, 3H), 2.25 (s, 6H), 2.04 (s, 3H), 1.89 (s, 6H), 1.78 (s, 3H). ¹³C NMR (125 MHz, CDCl₃) δ 139.22, 138.84, 137.10, 134.90, 134.25, 130.16, 129.48, 127.76, 126.21, 126.15, 125.74, 125.70, 53.44, 48.61, 21.09, 21.07, 20.54, 17.86, 9.51, 8.96. HRMS calcd for C₂₄H₃₀AgClN₂Na (M⁺+Na) 511.1041, found 511.1055.

Synthesis of 1-mesityl-3-(2,4,6-trimethylbenzyl)-imidazol-2-ylidene silver(I) chloride [Ag(NHC-4)Cl]

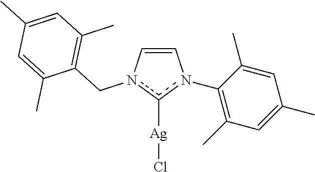

Following General Procedure C using NHC-4 the title compound was obtained as a white solid (78.5 mg, 85% yield). ¹H NMR (500 MHz, CDCl₃) δ 6.94 (s, 4H), 6.84 (s, 1H), 6.70 (s, 1H), 5.42 (s, 2H), 2.33 (s, 3H), 2.31 (s, 9H), 1.98 (s, 6H). ¹³C NMR (125 MHz, CDCl₃) δ 139.59, 139.14, 137.81, 135.42, 134.65, 129.83, 129.49, 127.74, 122.57, 119.79, 49.92, 21.07, 21.06, 20.03, 17.72. HRMS calcd for C₂₂H₂₆AgClN₂Na (M⁺+Na) 483.0728, found 483.0736.

Synthesis of 1-benzyl-3-(2,6-diisopropylphenyl)-4,5-dimethyl-imidazol-2-ylidene silver(I) chloride [Ag(NHC-5)Cl]

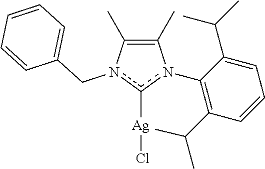

Following General Procedure C using NHC-5a the title compound was obtained as a white solid (83.3 mg, 85% yield). ¹H NMR (500 MHz, CDCl₃) δ 7.40 (t, J=7.8 Hz, 1H), 7.29-7.26 (m, 3H), 7.22-7.21 (d, J=7.8 Hz, 2H), 7.08 (s, 2H), 5.52 (s, 2H), 2.29-2.20 (m, 2H), 2.02 (s, 3H), 1.75 (s, 3H), 1.20 (d, J=7.2 Hz, 6H), 1.09 (d, J=7.2 Hz, 6H). ¹³C NMR (125 MHz, CDCl₃) δ 145.96, 135.77, 133.05, 130.54, 129.30, 129.08, 128.28, 128.00, 126.50, 125.28, 124.43, 53.46, 28.33, 25.44, 23.34, 9.59, 9.45. HRMS calcd for C₂₄H₃₀AgClN₂Na (M⁺+Na) 511.1041, found 511.1055.

Synthesis of allyl [1-(2,6-diisopropylphenyl)-4,5-dimethyl-3-(2,4,6-trimethylbenzyl)-1H-imidazol-2-ylidene] chloropalladium(II) [Pd(NHC-1)(allyl)Cl]

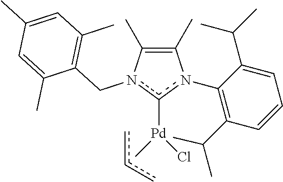

An oven-dried flask equipped with a stir bar was charged with NHC-HCl salt (NHC-1) (153.0 mg, 0.36 mmol, 1.2 equiv), placed under a positive pressure of argon and subjected to three evacuation/backfilling cycles under high vacuum. THF (2.0 mL) and LiHMDS (1.0 M in THF, 0.33 mL, 0.33 mmol, 1.1 equiv) were added and the resulting reaction mixture was stirred at room temperature for 2 h. After the indicated time, [{Pd(allyl)Cl}₂] (55.0 mg, 0.15 mmol, 0.5 equiv) in THF (1.0 mL) was added and the reaction mixture was stirred at room temperature for 15 h. After the indicated time, the reaction mixture was diluted with CH₂Cl₂ (10 mL) and filtered. The solution was collected and concentrated. The title product was obtained by trituration from hexanes as a yellow-white solid. Yield 87.5% (150 mg). Mp=205-206° C. ¹H NMR (500 MHz, CDCl₃) δ 7.38 (t, J=7.7 Hz, 1H), 7.24 (d, J=7.7 Hz, 1H), 7.21-7.17 (m, 1H), 6.79 (s, 2H), 5.80 (d, J=16.0 Hz, 1H), 5.64 (d, J=16.0 Hz, 1H), 4.88-4.72 (m, 1H), 4.05 (dd, J=7.4, 1.7 Hz, 1H), 2.95-2.87 (m, 3H), 2.63-2.57 (m, 1H), 2.22 (s, 3H), 2.21 (s, 6H), 1.80 (s, 3H), 1.77 (s, 3H), 1.49 (d, J=11.9 Hz, 1H), 1.31 (d, J=6.7 Hz, 3H), 1.21 (d, J=6.8 Hz, 3H), 1.08-1.01 (m, 6H). ¹³C NMR (126 MHz, CDCl₃) δ 182.14, 147.31, 146.37, 137.34, 137.16, 134.61, 130.07, 129.60, 129.56, 126.96 , 126.27, 124.13, 123.76, 114.25, 72.87, 50.47, 49.03, 28.00, 27.94, 24.95, 24.87, 24.66, 24.15, 20.86, 20.70, 9.76, 9.56. HRMS (ESI) m/z: [M-Cl]⁺ Calcd for C₃₀H₄₁N₂Pd 535.2311; Found 535.2309.

Synthesis of cinnamyl [1-(2,6-diisopropylphenyl)-4,5-dimethyl-3-(2,4,6-trimethylbenzyl)-1H-imidazol-2-ylidene] chloropalladium(II) [Pd(NHC-1)(cin)Cl]

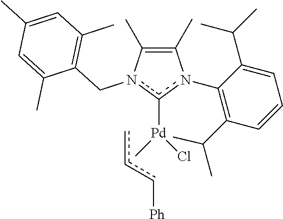

Following General Procedure D using NHC-1 the title compound was obtained as a yellow solid (49.2 mg, 76% yield). ¹H NMR (500 MHz, CDCl₃) δ 7.47 (t, J=7.8 Hz, 1H), 7.35-7.20 (m, 7H), 6.87 (s, 2H), 5.80 (d, J=15.8 Hz, 1H), 5.60 (d, J=15.8 Hz, 1H), 5.23-5.16 (m, 1H), 4.48 (d, J=12.8 Hz, 1H), 3.06-3.01 (m, 1H), 2.83 (d, J=7.2 Hz, 1H), 2.70-2.64 (m, 1H), 2.30 (s, 3H), 2.27 (s, 6H), 1.87 (s, 6H), 1.59 (d, J=12.8 Hz, 1H), 1.42 (d, J=7.2 Hz, 3H), 1.26 (d, J=7.2 Hz, 3H), 1.12-1.10 (m, 6H). ¹³C NMR (125 MHz, CDCl₃) δ 181.05, 147.38, 146.38, 137.85, 137.37, 137.04, 134.59, 130.28, 129.57, 129.48, 128.46, 127.07, 127.02, 126.80, 126.44, 124.14, 123.69, 108.51, 91.04, 50.11, 45.46, 27.94, 27.85, 25.02, 24.88, 24.66, 24.09, 20.80, 20.74, 20.60, 9.78, 9.60. HRMS calcd for C₃₆H₄₅PdClN₂Na (M⁺+Na) 669.2198, found 669.2199.

Synthesis of cinnamyl [1-mesityl-4,5-dimethyl-3-(2,4,6-trimethylbenzyl)-1H-imidazol-2-ylidene] chloropalladium(II) [Pd(NHC-3)(cin)Cl]

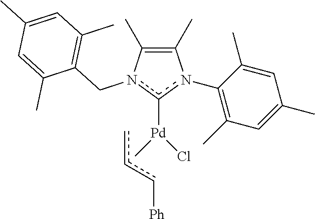

Following General Procedure D using NHC-3 the title compound was obtained as a yellow solid (45.4 mg, 75% yield). ¹H NMR (500 MHz, CDCl₃) δ 7.31-7.27 (m, 4H), 7.22-7.19 (m, 1H), 7.01 (s, 1H), 6.95 (s, 1H), 6.86 (s, 2H), 5.65 (d, J=15.8 Hz, 1H), 5.28 (d, J=15.8 Hz, 1H), 4.98-4.92 (m, 1H), 4.32 (d, J=12.8 Hz, 1H), 2.83 (d, J=7.2 Hz, 1H), 2.36 (s, 3H), 2.31 (s, 3H), 2.25 (s, 9H), 2.01 (s, 3H), 1.99 (s, 3H), 1.81 (s, 3H), 1.61 (d, J=7.2 Hz, 1H). ¹³C NMR (125 MHz, CDCl₃) δ 179.03, 138.50, 138.16, 137.42, 137.04, 136.83, 135.06, 135.00, 130.54, 129.46, 129.35, 128.40, 128.35, 126.99, 126.61, 126.39, 125.44, 108.85, 89.73, 48.33, 45.36, 21.11, 20.83, 20.82, 18.88, 17.97, 9.72, 8.91. HRMS calcd for C₃₃H₃₉PdClN₂Na (M⁺+Na) 627.1729, found 627.1736.

Synthesis of cinnamyl [1-(2,6-diisopropylphenyl)-3-(2,4,6-trimethylbenzyl)-1H-imidazol-2-ylidene] chloropalladium(II) [Pd(NHC-2)(cin)Cl]

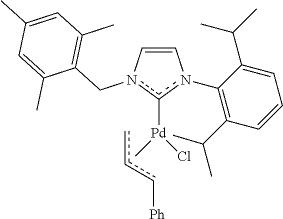

Following General Procedure D using NHC-2 the title compound was obtained as a yellow solid (43.4 mg, 70% yield). ¹H NMR (500 MHz, CDCl₃) δ 7.46 (t, J=7.8 Hz, 1H), 7.37-7.27 (m, 6H), 7.22-7.19 (m, 1H), 6.92 (s, 2H), 6.89 (d, J=1.6 Hz, 1H), 6.66 (d, J=1.6 Hz, 1H), 5.70 (d, J=15.8 Hz, 1H), 5.61 (d, J=15.8 Hz, 1H), 5.41-5.35 (m, 1H), 4.58 (d, J=12.8 Hz, 1H), 3.02-2.99 (m, 2H), 2.61-2.58 (m, 1H), 2.31 (s, 9H), 1.75 (d, J=12.8 Hz, 1H), 1.42 (d, J=7.2 Hz, 3H), 1.27 (d, J=7.2 Hz, 3H), 1.05 (d, J=7.2 Hz, 6H). ¹³C NMR (125 MHz, CDCl₃) δ 181.86, 147.00, 146.03, 138.10, 137.95, 137.77, 135.98, 129.81, 129.53, 129.46, 128.54, 128.30, 127.93, 127.21, 126.98, 123.93, 123.87, 123.41, 119.78, 108.87, 91.47, 50.22, 45.42, 28.19, 26.31, 26.07, 23.39, 22.83, 20.99, 20.04. HRMS calcd for C₃₄H₄₁PdClN₂Na (M⁺+Na) 641.1885, found 641.1892.

Synthesis of cinnamyl [1-mesityl-3-(2,4,6-trimethylbenzyl)-1H-imidazol-2-ylidene] chloropalladium(II) [Pd(NHC-4)(cin)Cl]

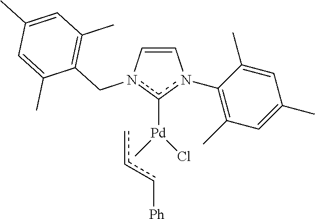

Following General Procedure D using NHC-4 the title compound was obtained as a yellow solid (40.4 mg, 70% yield). ¹H NMR (500 MHz, CDCl₃) δ 7.39 (d, J=7.4 Hz, 2H), 7.29-7.26 (m, 1H), 7.22-7.19 (m, 1H), 7.01 (s, 1H), 6.95 (s, 1H), 6.91 (s, 2H), 6.81 (d, J=1.6 Hz, 1H), 6.66 (d, J=1.6 Hz, 1H), 5.65 (d, J=15.8 Hz, 1H), 5.51 (d, J=15.8 Hz, 1H), 5.43-5.36 (m, 1H), 4.59 (d, J=12.8 Hz, 1H), 3.10 (d, J=6.8 Hz, 1H), 2.36 (s, 3H), 2.30 (s, 9H), 2.28 (s, 3H), 2.03 (s, 3H), 1.85 (d, J=12.8 Hz, 1H). ¹³C NMR (125 MHz, CDCl₃) δ 180.75, 138.80, 138.10, 137.93, 136.41, 136.32, 134.94, 129.42, 129.37, 129.27, 129.22, 128.49, 128.29, 127.98, 127.22, 126.90, 122.08, 120.36, 109.07, 90.92, 49.85, 45.36, 30.89, 21.10, 20.96, 20.12, 19.98, 18.72, 17.92. HRMS calcd for C₃₁H₃₅PdClN₂Na (M⁺+Na) 599.1416, found 599.1426.

Synthesis of cinnamyl [1-(2,6-diisopropylphenyl)-4,5-dimethyl-3-benzyl-1H-imidazol-2-ylidene] chloropalladium(II) [Pd(NHC-5)(cin)Cl]

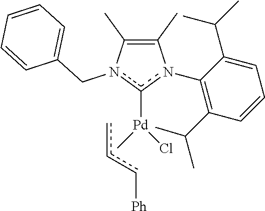

Following General Procedure D using NHC-5a the title compound was obtained as a yellow solid (40.0 mg, 66% yield). ¹H NMR (500 MHz, CDCl₃) δ 7.48 (t, J=7.8 Hz, 1H), 7.35-7.26 (m, 9H), 7.22-7.17 (m, 3H), 5.84 (d, J=15.8 Hz, 1H), 5.65 (d, J=15.8 Hz, 1H), 5.23-5.17 (m, 1H), 4.49 (d, J=12.8 Hz, 1H), 2.99-2.97 (m, 1H), 2.80 (d, J=7.2 Hz, 1H), 2.72-2.69 (m, 1H), 2.10 (s, 3H), 1.92 (s, 3H), 1.61 (d, J=12.8 Hz, 1H), 1.41 (d, J=7.2 Hz, 3H), 1.28 (d, J=7.2 Hz, 3H), 1.16-1.12 (m, 6H). ¹³C NMR (125 MHz, CDCl₃) δ 180.26, 147.08, 146.45, 138.20, 137.85, 134.82, 129.68, 128.49, 127.25, 127.18, 127.16, 126.91, 126.78, 126.07, 124.13, 123.85, 108.75, 91.24, 52.16, 45.96, 28.05, 24.78, 24.71, 24.46, 24.04, 9.89, 9.60. HRMS calcd for C₃₃H₃₉PdClN₂Na (M⁺+Na) 627.1729, found 627.1734.

Synthesis of 3-chloropyridine [1-(2,6-diisopropylphenyl)-4,5-dimethyl-3-(2,4,6-trimethylbenzyl)-1H-imidazol-2-ylidene] dichloropalladium(II) [Pd(NHC-1)(py)Cl₂]

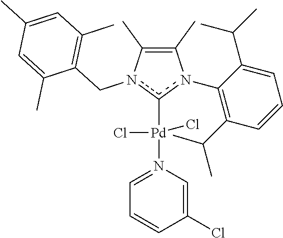

Following General Procedure E using NHC-1 the title compound was obtained as a yellow solid (29.9 mg, 88% yield). ¹H NMR (500 MHz, CDCl₃) δ 8.80 (s, 1H), 8.69 (d, J=5.2 Hz, 1H), 7.64 (d, J=7.8 Hz, 1H), 7.49 (t, J=7.8 Hz, 1H), 7.32 (d, J=7.8 Hz, 2H), 7.18 (dd, J=5.2 Hz, J=7.8 Hz, 1H), 6.89 (s, 2H), 6.17 (s, 2H), 2.89-2.86 (m, 2H), 2.35 (s, 6H), 2.31 (s, 3H), 1.80 (s, 3H), 1.69 (s, 3H), 1.47 (d, J=6.8 Hz, 6H), 1.06 (d, J=6.8 Hz, 6H). ¹³ C NMR (125 MHz, CDCl₃) δ 150.36, 149.32, 147.29, 146.68, 137.82, 137.63, 137.57, 132.60, 132.18, 130.14, 129.69, 129.50, 128.85, 127.22, 124.48, 124.33, 51.05, 28.21, 25.13, 24.88, 20.91, 20.75, 10.13, 9.78. HRMS calcd for C₃₂H₄₀PdCl₃N₃Na (M⁺+Na) 700.1215, found 700.1220.

Synthesis of 3-chloropyridine [1-mesityl-4,5-dimethyl-3-(2,4,6-trimethylbenzyl)-1H-imidazol-2-ylidene] dichloropalladium(II) [Pd(NHC-3)(py)Cl₂]

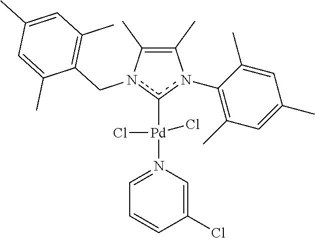

Following General Procedure E using NHC-3 the title compound was prepared as a yellow solid (27.1 mg, 85% yield). ¹H NMR (500 MHz, CDCl₃) δ 8.74 (s, 1H), 8.66 (d, J=5.2 Hz, 1H), 7.63 (d, J=7.8 Hz, 1H), 7.16 (dd, J=5.2 Hz, J=7.8 Hz, 1H), 7.01 (s, 2H), 6.88 (s, 2H), 6.04 (s, 2H), 2.35 (s, 9H), 2.30 (s, 3H), 2.22 (s, 6H), 1.73 (s, 3H), 1.70 (s, 3H). ¹³ C NMR (125 MHz, CDCl₃) δ 150.36, 149.36, 145.67, 138.96, 137.93, 137.68, 137.55, 136.66, 133.09, 132.09, 129.65, 129.20, 128.87, 127.87, 127.06, 124.39, 50.32, 21.21, 20.97, 20.92, 18.95, 9.82, 8.76. HRMS calcd for C₂₉H₃₄PdCl₃N₃Na (M⁺+Na) 658.0745, found 658.0756.

Synthesis of 3-chloropyridine [1-(2,6-diisopropylphenyl)-3-(2,4,6-trimethylbenzyl)-1H-imidazol-2-ylidene] dichloropalladium(H) [Pd(NHC-2)(py)Cl₂]

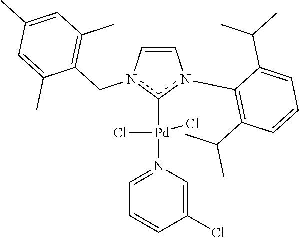

Following General Procedure E using NHC-2 the title compound was obtained as a yellow solid (26.7 mg, 82% yield). ¹H NMR (500 MHz, CDCl₃) δ 8.90 (s, 1H), 8.78 (d, J=5.2 Hz, 1H), 7.67 (d, J=7.8 Hz, 1H), 7.48 (t, J=7.8 Hz, 1H), 7.32 (d, J=7.8 Hz, 2H), 7.21 (dd, J=5.2 Hz, J=7.8 Hz, 1H), 6.98 (s, 2H), 6.82 (s, 1H), 6.54 (s, 1H), 6.02 (s, 2H), 2.93-2.87 (m, 2H), 2.41 (s, 6H), 2.33 (s, 3H), 1.41 (d, J=6.8 Hz, 6H), 1.02 (d, J=6.8 Hz, 6H). ¹³ C NMR (125 MHz, CDCl₃) δ 150.37, 149.71, 149.36, 147.02, 138.81, 138.76, 137.78, 134.43, 132.37, 130.30, 129.53, 127.89, 125.40, 124.62, 123.97, 119.43, 49.99, 28.42, 26.42, 23.21, 21.08, 20.19. HRMS calcd for C₃₀H₃₆PdCl₃N₃Na (M⁺+Na) 672.0902, found 672.0912.

Synthesis of 3-chloropyridine [1-mesityl-3-(2,4,6-trimethylbenzyl)-1H-imidazol-2-ylidene] dichloropalladium(II) [Pd(NHC-4)(py)Cl₂]

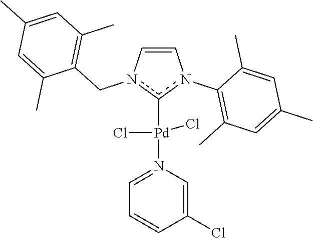

Following General Procedure E using NHC-4 the title compound was obtained as a yellow solid (45.7 mg, 75% yield). ¹H NMR (500 MHz, CDCl₃) δ 8.90 (d, J=1.8 Hz, 1H), 8.80 (d, J=5.2 Hz, 1H), 7.68 (d, J=7.8 Hz, 1H), 7.21 (dd, J=5.2 Hz, J=7.8 Hz, 1H), 7.01 (s, 2H), 6.96 (s, 2H), 6.76 (d, J=1.8 Hz, 1H), 6.53 (d, J=1.8 Hz, 1H), 5.98 (s, 2H), 2.38 (s, 6H), 2.36 (s, 3H), 2.32 (s, 3H), 2.25 (s, 6H), 2.17 (s, 9H). ¹³C NMR (125 MHz, CDCl₃) δ 150.38, 149.39, 148.82, 139.19, 138.77, 137.77, 136.32, 134.83, 132.32, 129.48, 129.20, 127.71, 124.54, 123.63, 120.14, 49.67, 21.16, 21.05, 20.10, 18.89. HRMS calcd for C₂₇H₃₀PdClN₂Na (M⁺+Na) 630.0432, found 630.0436.

3-chloropyridine [1-(2,6-diisopropylphenyl)-4,5-dimethyl-3-benzyl-1H-imidazol-2-ylidene] dichloropalladium(II) [Pd(NHC-5)(py)Cl₂]

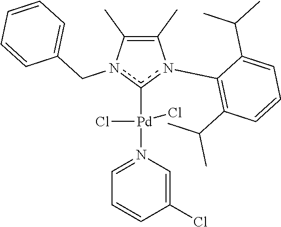

Following General Procedure E using NHC-5a the title compound was obtained as a yellow solid (40.0 mg, 66% yield). ¹H NMR (500 MHz, CDCl₃) δ 8.82 (d, J=1.8 Hz, 1H), 8.71 (d, J=5.2 Hz, 1H), 7.63 (d, J=7.8 Hz, 1H), 7.52-7.50 (m, 2H), 7.42-7.31 (m, 5H), 7.17-7.15 (m, 1H), 6.12 (s, 2H), 2.89-2.84 (m, 2H), 1.98 (s, 3H), 1.83 (s, 3H), 1.48 (d, J=6.8 Hz, 6H), 1.07 (d, J=6.8 Hz, 6H). ¹³C NMR (125 MHz, CDCl₃) δ 150.35, 149.35, 147.50, 146.91, 137.62, 136.19, 132.49, 132.19, 130.22, 129.79, 128.83, 127.81, 127.56, 126.75, 124.51, 124.47, 53.68, 28.27, 25.26, 25.01, 10.27, 9.75. HRMS calcd for C₂₉H₃₄PdCl₃N₃Na (M⁺+Na) 658.0745, found 658.0754.

### Example 5

### Steric Parameters of NHC Complexes

The catalysts of the present disclosure, comprising Cu-NHC, Au-NHC, and Ag-NHC catalysts have been characterized by a number of methods. Unexpectedly, a variation in the buried volume percentage (% V_{bur}) and steric maps of [M(NHC)Cl] complexes were observed as a function of both N-wingtip substitution and backbone modification. A representative % V_{bur} of complex [Au(NHC-1)Cl] is 38.3%, which is between that of IPr (% V_{bur}=45.4%) and IMes (% V_{bur}=36.5%). The N-wingtip is fully flexible depending on the scaffold. Thus, two isomers were crystallized for representative complex [Au(NHC-3)Cl], having % V_{bur}=34.5% (anti-conformation) and % V_{bur}=47.0% (syn-conformation), indicating that the N-wingtip bond is fully flexible and can provide accommodation to the steric environment, which is not possible using IPr or IMes ligands. [Cu(NHC-1)Cl], having one of the largest % V_{bur} values of the present disclosure (% V_{bur}=52.7%), exceeds that of IPr, while accommodating full N-flexibility.

### Example 6

### Catalytic Activity of Unsymmetrical NHC Complexes (Borylation)

The catalytic activity of the novel Cu(I)-NHC catalysts of the present disclosure has been demonstrated in both β-hydroboration and carboboration reactions. Using the catalysts described herein, the reactions may be conducted with a catalyst loading of 0.01 to 0.02 mol %, open to air at room temperature to afford tri-substituted vinylboronates. For the synthesis of tetra-substituted vinylboronates, a catalyst loading of 2.0 mol % is preferred.

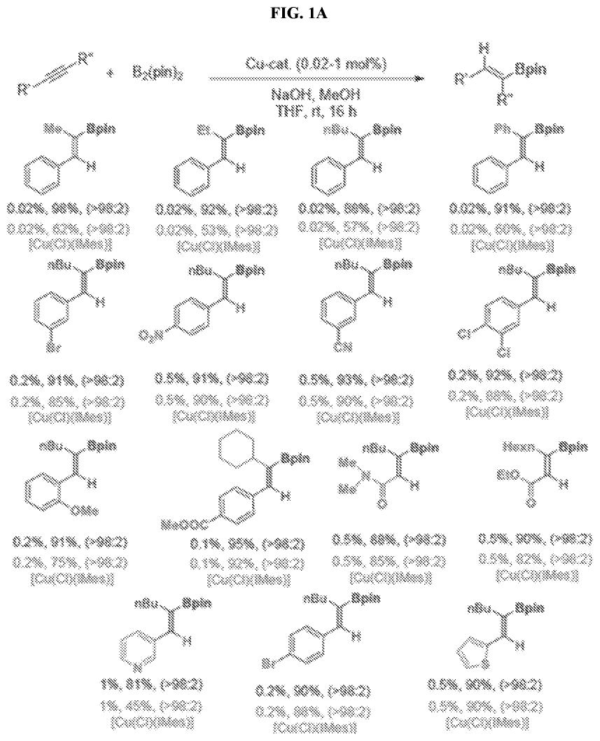

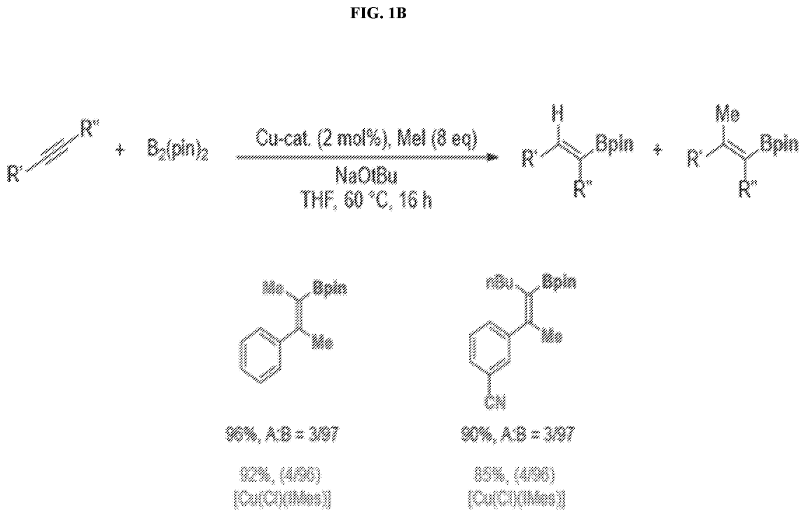

The scope of the borylation reactions have been investigated and high catalytic activity has been demonstrated for numerous examples, with excellent functional group tolerance (e.g. halides, nitro, cyano, and polar heterocycles). Representative reactions (e.g. hydroboration and carboboration) were performed using [Cu(NHC-1)Cl] and compared to the standard [Cu(IMes)Cl], a known symmetrical NHC catalyst (FIGS. 1A-1B).

For the β-borylation of 1-phenyl-1-propyne using B₂pin₂ (Scheme 3), catalytic performance studies revealed the following order of catalytic activity for Cu(I)-NHCs: [Cu(NHC-1)Cl]>[Cu(NHC-3)Cl]>[Cu(NHC-5)Cl]>[Cu(NHC-2)Cl]>[Cu(NHC-4)Cl] (Table 2).

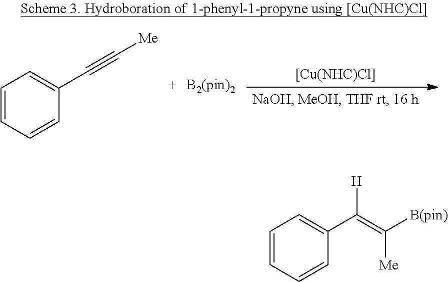

TABLE 2
Hydroboration of 1-phenyl-1-propyne
with B₂(pin)₂ using [Cu(NHC)Cl]^{a}

| | catalyst loading | yield |
|---|---|---|
| NHC catalyst | (mol %) | (%) |
| [Cu(NHC-1)Cl] | 0.01% | 50% |
| [Cu(NHC-1)Cl] | 0.02% | 98% |
| [Cu(NHC-2)Cl] | 0.01% | trace |
| [Cu(NHC-2)Cl] | 0.02% | 40% |
| [Cu(NHC-3)Cl] | 0.01% | 33% |
| [Cu(NHC-3)Cl] | 0.02% | 94% |
| [Cu(NHC-4)Cl] | 0.01% | trace |
| [Cu(NHC-4)Cl] | 0.02% | 36% |
| [Cu(NHC-5)Cl] | 0.01% | 25% |
| [Cu(NHC-5)Cl] | 0.02% | 92% |

^{a}1-phenyl-1-propyne (1.0 equiv), B₂(pin)₂ (1.2 equiv), NaOH (0.05 equiv), MeOH (0.05M), THF (0.5M), [Cu(NHC)Cl] (0.01-0.02 mol %).

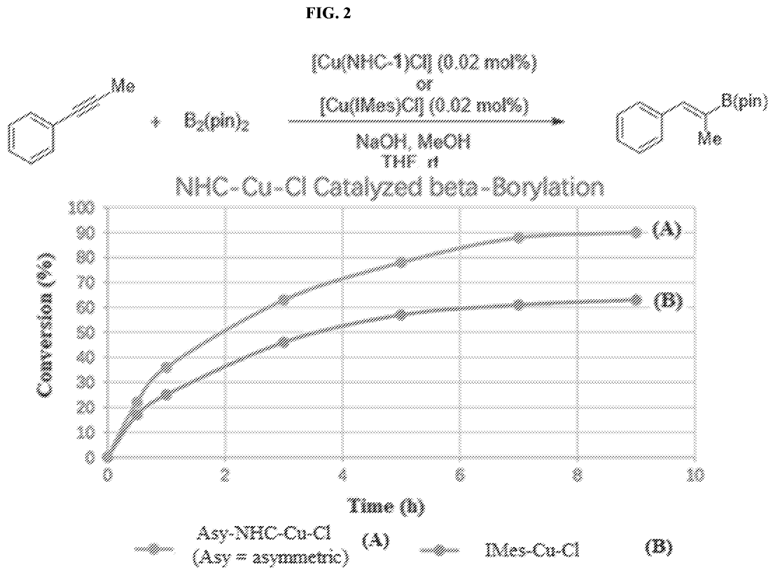

Kinetic studies were further used to evaluate the performance of [Cu(NHC-1)Cl] with respect to the most reactive catalyst known in the art (i.e. [Cu(IMes)Cl]). The catalyst of the present disclosure showed a better reactivity profile than [Cu(IMes)Cl], clearly demonstrating the superiority of the catalysts described herein for Cu-catalyzed hydrofunctionalizations (FIG. 2).

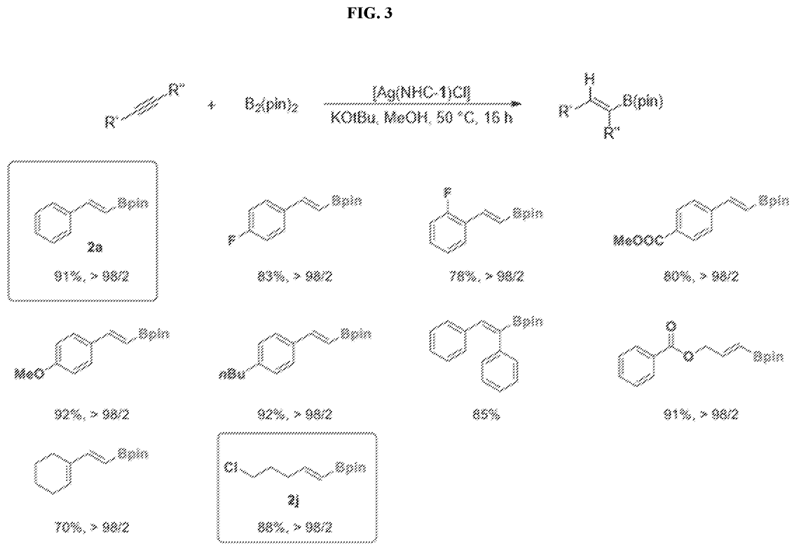

In addition to Cu(I)-NHCs, Ag(I)-NHCs also demonstrate high catalytic activity for borylation reactions, including but not limited to the hydroboration of phenylacetylene (Scheme 4). [Ag(NHC-1)Cl], [Ag(NHC-2)Cl], [Ag(NHC-3)Cl], [Ag(NHC-4)Cl], and [Ag(NHC-5)Cl] demonstrated superior catalytic activity in the hydroboration as compared to [Ag(IMes)Cl] (Table 3). Thus, the high catalytic performance of the catalysts described herein may be attributed to the unsymmetrical substitution, rather than the specific substitution patterns of the unsymmetrical or particular Group XI transition metal utilized in the NHCs. Furthermore, using the [Ag(NHC)Cl] catalysts described herein, a broad substrate scope was observed with regard to substitution of the acetylene in the hydroboration (FIG. 3).

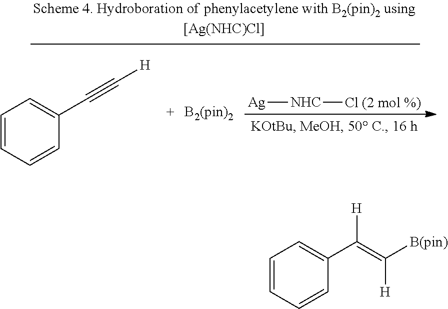

TABLE 3
Hydroboration of phenylacetylene using [Ag(NHC)Cl]^{a}

| | Yield | trans/cis |
|---|---|---|
| NHC catalyst | (%) | product ratio |
| [Ag(NHC-1)Cl] | 91% | 98/2 |
| [Ag(NHC-2)Cl] | 85% | 98/2 |
| [Ag(NHC-3)Cl] | 78% | 98/2 |
| [Ag(NHC-4)Cl] | 80% | 98/2 |
| [Ag(NHC-5)Cl] | 77% | 98/2 |
| [Ag(IMes)Cl] | 68% | 98/2 |

^{a}phenylacetylene (1.0 equiv), B₂(pin)₂ (1.2 equiv), KOtBu (0.05 equiv), MeOH (0.05M), [Ag(NHC)Cl] (2 mol %).

### Example 7

### Catalytic Activity of Unsymmetrical NHC Complexes (Hydration)

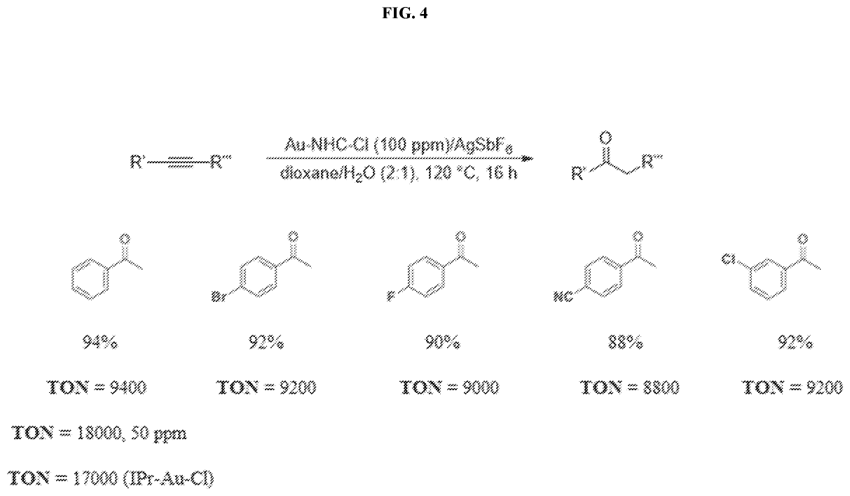

The catalytic activity of the novel Au(I)-NHC catalysts of the present disclosure have been demonstrated in hydration reactions of both alkynes and nitriles. [Au(NHC-1)Cl] matched or exceeded the performance of the known catalyst [Au(IPr)Cl], which is among the most reactive catalysts discovered to date for this class of alkyne hydrations (FIG. 4).

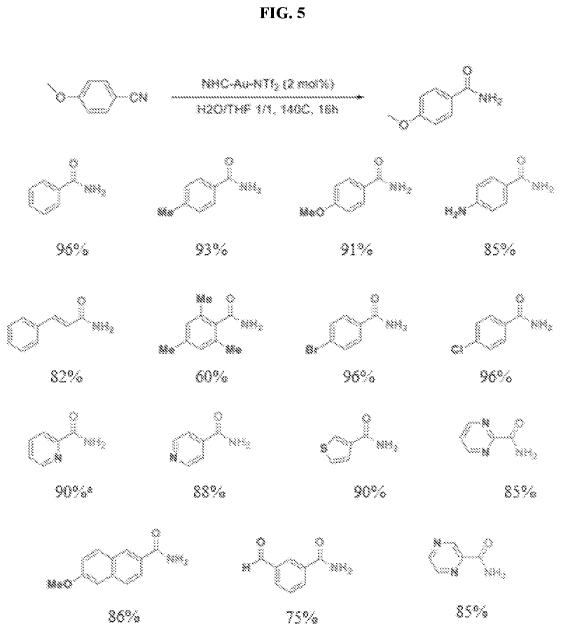

Furthermore, the hydration of nitriles using [Ag(NHC-1)NTf₂], prepared from the corresponding chloride [Ag(NHC-1)Cl], demonstrating that the high catalytic performance of the scaffold translates to additional counter anions and transformations (FIG. 5).

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

### ENUMERATED EMBODIMENTS

The following exemplary embodiments are provided, the numbering of which is not to be construed as designating levels of importance:

### Embodiment 1 provides a compound of formula (I):

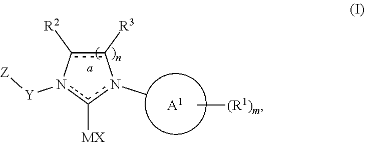

wherein:
M is a group XI (11) transition metal;
X is a counter anion;
Y is C(R^{a})(R^{a});
Z is selected from the group consisting of optionally substituted phenyl, optionally substituted naphthyl, optionally substituted C₄-C₁₀ heteroaryl, —C(═O)C₆-C₁₀ aryl, —C(═O)C₁-C₆ alkyl, —C(═O)OR^{a}, CN, and —C(═O)N(R^{a})(R^{b}),
   wherein each optional substituent in Z is independently selected from the group consisting of halogen, hydroxy, amino, NO₂, CN, —C(═O)OR^{a}, N(R^{a})(R^{b}), C₁-C₃ alkyl, C₁-C₃ haloalkyl, C₂-C₆ alkenyl, benzyl, phenyl, and naphthyl, and C₄-C₁₀ heteroaryl;
each occurrence of [symbol] is a single or double bond,
   wherein no more than one [symbol] bonding a C atom and a N atom is a double bond;
A¹ is selected from the group consisting of phenyl, naphthyl, and C₄-C₁₀ heteroaryl;
each occurrence of R¹ is independently selected from the group consisting of OR^{a}, N(R^{a})(R^{b}), optionally substituted C₁-C₁₂ alkyl, optionally substituted C₃-C₁₂ cycloalkyl, optionally substituted C₂-C₁₀ heterocycloalkyl, optionally substituted C₂-C₆ alkenyl, optionally substituted benzyl, optionally substituted phenyl, optionally substituted naphthyl, optionally substituted C₄-C₁₀ heteroaryl,
   wherein each optional substituent in R¹ is independently selected from the group consisting of C₁-C₃ alkyl, C₂-C₆ alkenyl, benzyl, phenyl, and naphthyl, and C₄-C₁₀ heteroaryl;
R² and R³ are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
   R² and R³ may combine with the ring to which they are attached to form a C₄-C₁₂ cycloalkyl, C₆-C₁₀ aryl, or C₄-C₁₀ heteroaryl,
   wherein each optional substituent in R² and R³ is independently at least one substituent selected from the group consisting of C₁-C₃ alkyl, C₂-C₆ alkenyl, phenyl, naphthyl, C₄-C₁₀ heteroaryl, OR^{b}, N(R^{a})(R^{b}), CN, CF₃, OCF₃, C(═O)R^{b}, N(R)S(═O)₂R^{b}, C(═O)N(R^{a})(R^{b}), and C(═O)OR^{b};
each occurrence of R^{a} is independently selected from the group consisting of H, optionally substituted C₁-C₃ alkyl, optionally substituted C₃-C₁₂ cycloalkyl, optionally substituted C₂-C₁₀ heterocycloalkyl, optionally substituted C₆-C₁₀ aryl, and optionally substituted C₄-C₁₀ heteroaryl,
   wherein each optional substituent in R^{a} is independently at least one substituent selected from the group consisting of C₁-C₃ alkyl, halogen, CN, NO₂, OR^{b}, OCF₃, and CF₃;
each occurrence of R^{b} is independently selected from the group consisting of H, C₁-C₆ alkyl, C₂-C₆ alkenyl, benzyl, phenyl, naphthyl, and C₄-C₁₀ heteroaryl;
m is an integer selected from the group consisting of 0, 1, 2, 3, 4, and 5; and
n is an integer which is either 1 or 2.

Embodiment 2 provides the compound of Embodiment 1, wherein Y is —CH₂—.

Embodiment 3 provides the compound of any of Embodiments 1-2, wherein bond a is a double bond.

Embodiment 4 provides the compound of any of Embodiments 1-3, wherein m is 2 or 3.

Embodiment 5 provides the compound of any of Embodiments 1-4, wherein each R¹ is independently selected from the group consisting of methyl, ethyl, n-propyl, isopropyl, n-butyl, sec-butyl, isobutyl, tert-butyl, cyclopropyl, cyclobutyl, cyclopentyl, cyclohexyl, cycloheptyl, cyclooctyl, —CH(CH₂CH₃)₂, —CH(CH(CH₃)₂)₂, —CH(CH₂CH₂CH₂CH₃)₂, phenyl, CHPh₂, —OCH₃, and —O(CH(CH₃)₂).

Embodiment 6 provides the compound of any of Embodiments 1-5, wherein n is 1.

Embodiment 7 provides the compound of any of Embodiments 1-6, which is a compound of formula (II):

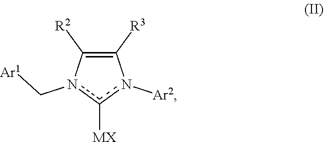

wherein:
X is selected from the group consisting of halogen, OS(═O)₂R^{c}, OC(═O)R^{c}, N(C(═O)R^{c})₂, tetracoordinate boronate, hexacoordinate phosphorus, and H;
each occurrence of R^{c} is independently selected from the group consisting of C₁-C₃ alkyl, C₁-C₃ haloalkyl, and phenyl optionally substituted with at least one substituent selected from the group consisting of C₁-C₃ alkyl and halogen;
Ar¹ and Ar² are each independently selected from the group consisting of optionally substituted phenyl, optionally substituted naphthyl, and optionally substituted C₄-C₁₀ heteroaryl,
   wherein each optional substituent in Ar¹ and Ar² is independently at least one substituent selected from the group consisting of OR^{a}, N(R^{a})(R^{b}), C₁-C₁₂ alkyl, C₃-C₁₂ cycloalkyl, C₂-C₁₀ heterocycloalkyl, C₁-C₃ haloalkyl, benzyl, and —CH₂—(C₆-C₁₀ heteroaryl), and
   wherein the optional benzyl and —CH₂—(C₆-C₁₀ heteroaryl) substituents in Ar¹ and Ar² are independently optionally substituted with at least one substituent selected from the group consisting of C₁-C₃ alkyl, C₆-C₁₀ aryl, and C₆-C₁₀ heteroaryl; and
R² and R³ are each independently selected from the group consisting of H, C₁-C₆ alkyl, C₂-C₆ alkenyl, phenyl, and naphthyl, C₄-C₁₀ heteroaryl.

Embodiment 8 provides the compound of any of Embodiments 1-7, wherein R² and R³ are each independently selected from the group consisting of H, methyl, ethyl, n-propyl, isopropyl, n-butyl, sec-butyl, isobutyl, and tert-butyl.

Embodiment 9 provides the compound of any of Embodiments 1-8, wherein R² and R³ are each independently H or Me.

Embodiment 10 provides the compound of any of Embodiments 1-9, wherein R² and R³ are identical.

Embodiment 11 provides the compound of any of Embodiments 1-10, wherein each of Ar¹ and Ar² is independently selected from the group consisting of phenyl,

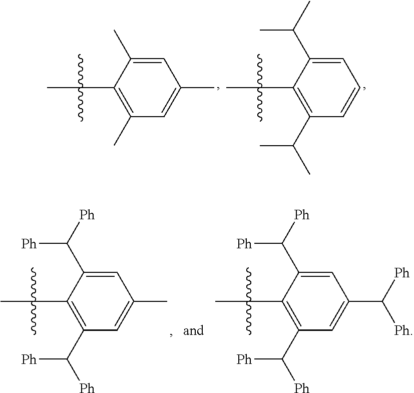

Embodiment 12 provides the compound of any of Embodiments 1-11, wherein M is selected from the group consisting of Cu, Ag, and Au.

Embodiment 13 provides the compound of any of Embodiments 1-12, wherein X is selected from the group consisting of Cl, Br, triflate (OTf), bistriflamide (NTf₂), tosylate (OTs), trifluoroacetate (TFA), BF₄, PF₆, and H.

Embodiment 14 provides the compound of any of Embodiments 1-13, wherein X is selected from the group consisting of Cl and NTf₂.

Embodiment 15 provides the compound of any of Embodiments 1-14, which is selected from the group consisting of:
1-(2,6-diisopropylphenyl)-4,5-dimethyl-3-(2,4,6-trimethylbenzyl)-imidazol-2-ylidene copper(I) chloride;
1-(2,6-diisopropylphenyl)-3-(2,4,6-trimethylbenzyl)-imidazol-2-ylidene copper(I) chloride;
1-mesityl-4,5-dimethyl-3-(2,4,6-trimethylbenzyl)-imidazol-2-ylidene copper(I) chloride;
1-mesityl-3-(2,4,6-trimethylbenzyl)-imidazol-2-ylidene copper(I) chloride;
1-benzyl-3-(2,6-diisopropylphenyl)-4,5-dimethyl-imidazol-2-ylidene copper(I) chloride;
1-(2,6-diisopropylphenyl)-4,5-dimethyl-3-(2,4,6-trimethylbenzyl)-imidazol-2-ylidene gold(I) bistriflimide;
1-(2,6-diisopropylphenyl)-4,5-dimethyl-3-(2,4,6-trimethylbenzyl)-imidazol-2-ylidene gold(I) chloride;
1-(2,6-diisopropylphenyl)-3-(2,4,6-trimethylbenzyl)-imidazol-2-ylidene gold(I) chloride;
1-mesityl-4,5-dimethyl-3-(2,4,6-trimethylbenzyl)-imidazol-2-ylidene gold(I) chloride;
1-mesityl-3-(2,4,6-trimethylbenzyl)-imidazol-2-ylidene gold(I) chloride;
1-benzyl-3-(2,6-diisopropylphenyl)-4,5-dimethyl-imidazol-2-ylidene gold(I) chloride;
1-(2,6-diisopropylphenyl)-4,5-dimethyl-3-(2,4,6-trimethylbenzyl)-imidazol-2-ylidene silver(I) chloride;
1-(2,6-diisopropylphenyl)-3-(2,4,6-trimethylbenzyl)-imidazol-2-ylidene silver(I) chloride;
1-mesityl-4,5-dimethyl-3-(2,4,6-trimethylbenzyl)-imidazol-2-ylidene silver(I) chloride;
1-mesityl-3-(2,4,6-trimethylbenzyl)-imidazol-2-ylidene silver(I) chloride; and 1-benzyl-3-(2,6-diisopropylphenyl)-4,5-dimethyl-imidazol-2-ylidene silver(I) chloride.

Embodiment 16 provides a method of promoting a reaction between an alkyne and a borylation reagent, the method comprising contacting the alkyne and the borylation reagent in the presence of a base, a protic solvent or an electrophile, and the compound of any of Embodiments 1-15.

Embodiment 17 provides the method of Embodiment 16, wherein the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 18 provides the method of any of Embodiments 16-17, wherein the alkyne is a terminal alkyne.

Embodiment 19 provides the method of any of Embodiments 16-18, wherein the borylation reagent is a diboronic ester.

Embodiment 20 provides the method of any of Embodiments 16-19, wherein the diboronic ester is B₂(pin)₂.

Embodiment 21 provides the method of any of Embodiments 16-20, wherein the compound of any of Embodiments 1-15 is present in an amount of ranging from about 0.02 to about 2.0 mol %.

Embodiment 22 provides the method of any of Embodiments 16-21, wherein the protic solvent is MeOH.

Embodiment 23 provides the method of any of Embodiments 16-22, wherein the base is selected from the group consisting of NaOH and NaOtBu. Embodiment 24 provides the method of any of Embodiments 16-23, wherein the contacting is in the presence of an aprotic solvent.

Embodiment 25 provides the method of Embodiment 24, wherein the aprotic solvent is tetrahydrofuran.

Embodiment 26 provides the method of any of Embodiments 16-25, wherein the electrophile is selected from the group consisting of C₁-C₁₂ haloalkyl, C₆-C₁₂ haloaralkyl, C₆-C₁₀ aryl halide, C₄-C₁₀ heteroaryl halide, C₁-C₁₂alkyl-(C═O)Cl, C₆-C₁₀ aryl-(C═O)Cl, C₄-C₁₀ heteroaryl-(C═O)Cl, and CO₂.

Embodiment 27 provides the method of any of Embodiments 16-26, wherein the electrophile is MeI.

Embodiment 28 provides the method of any of Embodiments 16-27, wherein the contacting occurs at a temperature of about 60° C.

Embodiment 29 provides a method of promoting hydration of a first reagent, the method comprising contacting the first reagent and water in the presence of the compound of any of Embodiments 1-15.

Embodiment 30 provides the method of Embodiment 29, wherein the first reagent comprises an alkyne.

Embodiment 31 provides the method of any of Embodiments 29-30, wherein the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 32 provides the method of any of Embodiments 29-31, wherein the contacting is in the presence of a Lewis acid.

Embodiment 33 provides the method of Embodiment 32, wherein the Lewis acid is selected from the group consisting of AgNTf₂, AgOAc, AgOTf, NaBArF, KB(C₆F₅)₄, and AgSbF₆.

Embodiment 34 provides the method of any of Embodiments 29-33, wherein the compound of any of Embodiments 1-15 is present in an amount of about 100 ppm.

Embodiment 35 provides the method of any of Embodiments 29-34, wherein the contacting is in the presence of a solvent. Embodiment 36 provides the method of Embodiment 35, wherein the solvent is 1,4-dioxane.

Embodiment 37 provides the method of any of Embodiments 35-36, wherein the solvent and water have a ratio of about 2:1.

Embodiment 38 provides the method of any of Embodiments 29-37, wherein the contacting occurs at a temperature of about 120° C.

Embodiment 39 provides the method of Embodiment 29, wherein the first reagent is selected from the group consisting of optionally substituted C₁-C₁₂ alkyl nitrile, optionally substituted C₂-C₆ alkenyl nitrile, optionally substituted C₆-C₁₀ aryl nitrile, optionally substituted C₄-C₁₀ heteroaryl nitrile, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 40 provides the method of Embodiment 39, wherein the contacting is in the presence of a solvent.

Embodiment 41 provides the method of any of Embodiments 39-40, wherein the solvent is tetrahydrofuran.

Embodiment 42 provides the method of any of Embodiments 39-41, wherein the solvent and water have a ratio of about 1:1.

Embodiment 43 provides the method of any of Embodiments 39-42, wherein the contacting occurs at a temperature of about 140° C.

Embodiment 44 provides a compound of formula (III):

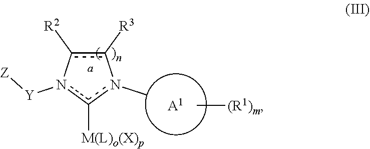

wherein:
M is a group X (10) transition metal;
L is a ligand of M, wherein each occurrence of L can be the same or different;
X is a counter anion;
Y is C(R^{a})(R^{a});
Z is selected from the group consisting of optionally substituted phenyl, optionally substituted naphthyl, optionally substituted C₄-C₁₀ heteroaryl, —C(═O)C₆-C₁₀ aryl, —C(═O)C₁-C₆ alkyl, —C(═O)OR^{a}, CN, and —C(═O)N(R^{a})(R^{b}),
   wherein each optional substituent in Z is independently selected from the group consisting of halogen, hydroxy, amino, NO₂, CN, —C(═O)OR^{a}, N(R^{a})(R^{b}), C₁-C₃ alkyl, C₁-C₃ haloalkyl, C₂-C₆ alkenyl, benzyl, phenyl, and naphthyl, and C₄-C₁₀ heteroaryl;
each occurrence of [symbol] is a single or double bond,
   wherein no more than one [symbol] bonding a C atom and a N atom is a double bond;
A¹ is selected from the group consisting of phenyl, naphthyl, and C₄-C₁₀ heteroaryl;
each occurrence of R¹ is independently selected from the group consisting of OR^{a}, N(R^{a})(R^{b}), optionally substituted C₁-C₁₂ alkyl, optionally substituted C₃-C₁₂ cycloalkyl, optionally substituted C₂-C₁₀ heterocycloalkyl, optionally substituted C₂-C₆ alkenyl, optionally substituted benzyl, optionally substituted phenyl, optionally substituted naphthyl, optionally substituted C₄-C₁₀ heteroaryl,
   wherein each optional substituent in R¹ is independently selected from the group consisting of C₁-C₃ alkyl, C₂-C₆ alkenyl, benzyl, phenyl, and naphthyl, and C₄-C₁₀ heteroaryl;
R² and R³ are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
   R² and R³ may combine with the ring to which they are attached to form a C₄-C₁₂ cycloalkyl, C₆-C₁₀ aryl, or C₄-C₁₀ heteroaryl,
   wherein each optional substituent in R² and R³ is independently at least one substituent selected from the group consisting of C₁-C₃ alkyl, C₂-C₆ alkenyl, phenyl, naphthyl, C₄-C₁₀ heteroaryl, OR^{b}, N(R^{a})(R^{b}), CN, CF₃, OCF₃, C(═O)R^{b}, N(R)S(═O)₂R^{b}, C(═O)N(R^{a})(R^{b}), and C(═O)OR^{b};
each occurrence of R^{a} is independently selected from the group consisting of H, optionally substituted C₁-C₃ alkyl, optionally substituted C₃-C₁₂ cycloalkyl, optionally substituted C₂-C₁₀ heterocycloalkyl, optionally substituted C₆-C₁₀ aryl, and optionally substituted C₄-C₁₀ heteroaryl,
   wherein each optional substituent in R^{a} is independently at least one substituent selected from the group consisting of C₁-C₃ alkyl, halogen, CN, NO₂, OR^{b}, OCF₃, and CF₃;
each occurrence of R^{b} is independently selected from the group consisting of H, C₁-C₆ alkyl, C₂-C₆ alkenyl, benzyl, phenyl, naphthyl, and C₄-C₁₀ heteroaryl;
m is an integer selected from the group consisting of 0, 1, 2, 3, 4, and 5;
n is an integer which is either 1 or 2;
o is an integer selected from the group consisting of 1 and 2; and
p is an integer selected from the group consisting of 1 and 2.

Embodiment 45 provides the compound of Embodiment 44, wherein Y is —CH₂—.

Embodiment 46 provides the compound of Embodiment 44 or 45, wherein bond a is a double bond.

Embodiment 47 provides the compound of any one of Embodiments 44-46, wherein m is 2 or 3.

Embodiment 48 provides the compound of any one of Embodiments 44-47, wherein n is 1.

Embodiment 49 provides the compound of any one of Embodiments 44-48, wherein o is 1.

Embodiment 50 provides the compound of any one of Embodiments 44-49, wherein p is 2.

Embodiment 51 provides the compound of any one of Embodiments 44-50, wherein each R¹ is independently selected from the group consisting of methyl, ethyl, n-propyl, isopropyl, n-butyl, sec-butyl, isobutyl, tert-butyl, cyclopropyl, cyclobutyl, cyclopentyl, cyclohexyl, cycloheptyl, cyclooctyl, —CH(CH₂CH₃)₂, —CH(CH(CH₃)₂)₂, —CH(CH₂CH₂CH₂CH₃)₂, phenyl, CHPh₂, —OCH₃, and —O(CH(CH₃)₂).

Embodiment 52 provides the compound of Embodiment 44, which is a compound of formula (IV):

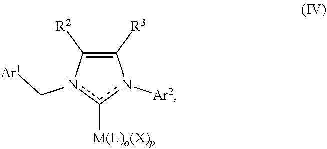

wherein:
X is selected from the group consisting of halogen, OS(═O)₂R^{c}, OC(═O)R^{c}, N(C(═O)R^{c})₂, tetracoordinate boronate, hexacoordinate phosphorus, H, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl;
L optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl;
each occurrence of R^{c} is independently selected from the group consisting of C₁-C₃ alkyl, C₁-C₃ haloalkyl, and phenyl optionally substituted with at least one substituent selected from the group consisting of C₁-C₃ alkyl and halogen;
Ar¹ and Ar² are each independently selected from the group consisting of optionally substituted phenyl, optionally substituted naphthyl, and optionally substituted C₄-C₁₀ heteroaryl,
   wherein each optional substituent in Ar¹ and Ar² is independently at least one substituent selected from the group consisting of OR^{a}, N(R^{a})(R^{b}), C₁-C₁₂ alkyl, C₃-C₁₂ cycloalkyl, C₂-C₁₀ heterocycloalkyl, C₁-C₃ haloalkyl, benzyl, and —CH₂—(C₆-C₁₀ heteroaryl), and
   wherein the optional benzyl and —CH₂—(C₆-C₁₀ heteroaryl) substituents in Ar¹ and Ar² are independently optionally substituted with at least one substituent selected from the group consisting of C₁-C₃ alkyl, C₆-C₁₀ aryl, and C₆-C₁₀ heteroaryl; and
R² and R³ are each independently selected from the group consisting of H, C₁-C₆ alkyl, C₂-C₆ alkenyl, phenyl, and naphthyl, C₄-C₁₀ heteroaryl.

Embodiment 53 provides the compound of Embodiment 52, wherein X is selected from the group consisting of Cl, Br, tosylate (OTs), mesylate (OMs), triflate (OTf), bistriflamide (NTf₂), allyl anion (i.e., vinylmethanide), and allylbenzene anion (i.e., 3-phenylpropen-3-ide and/or 1-phenylpropen-3-ide).

Embodiment 54 provides the compound of Embodiment 52 or 53, wherein L is selected from the group consisting of Cl and 3-chloropyridine.

Embodiment 55 provides the compound of any one of Embodiments 52-54, wherein R² and R³ are each independently selected from the group consisting of H, methyl, ethyl, n-propyl, isopropyl, n-butyl, sec-butyl, isobutyl, and tert-butyl.

Embodiment 56 provides the compound of Embodiment 55, wherein R² and R³ are each independently H or Me.

Embodiment 57 provides the compound of any one of Embodiments 52-56, wherein R² and R³ are identical.

Embodiment 58 provides the compound of any one of Embodiments 52-56, wherein each of Ar¹ and Ar² is independently selected from the group consisting of phenyl,

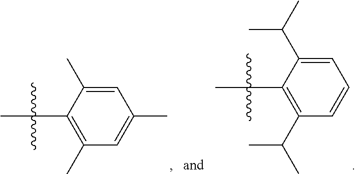

Embodiment 59 provides the compound of any one of Embodiments 52-58, wherein M is selected from the group consisting of Pd, Ni, and Pt.

Embodiment 60 provides the compound of Embodiment 59, wherein M is Pd.

Embodiment 61 provides the compound of Embodiment 44, which is selected from the group consisting of:
allyl [1-(2,6-diisopropylphenyl)-4,5-dimethyl-3-(2,4,6-trimethylbenzyl)-1H-imidazol-2-ylidene] chloropalladium(II);
cinnamyl [1-(2,6-diisopropylphenyl)-4,5-dimethyl-3-(2,4,6-trimethylbenzyl)-1H-imidazol-2-ylidene] chloropalladium(II);
cinnamyl [1-mesityl-4,5-dimethyl-3-(2,4,6-trimethylbenzyl)-1H-imidazol-2-ylidene] chloropalladium(II);
cinnamyl [1-(2,6-diisopropylphenyl)-3-(2,4,6-trimethylbenzyl)-1H-imidazol-2-ylidene] chloropalladium(II);
cinnamyl [1-mesityl-3-(2,4,6-trimethylbenzyl)-1H-imidazol-2-ylidene] chloropalladium(II);
cinnamyl [1-(2,6-diisopropylphenyl)-4,5-dimethyl-3-benzyl-1H-imidazol-2-ylidene] chloropalladium(II);
3-chloropyridine [1-(2,6-diisopropylphenyl)-4,5-dimethyl-3-(2,4,6-trimethylbenzyl)-1H-imidazol-2-ylidene] dichloropalladium(II);
3-chloropyridine [1-mesityl-4,5-dimethyl-3-(2,4,6-trimethylbenzyl)-1H-imidazol-2-ylidene] dichloropalladium(II);
3-chloropyridine [1-(2,6-diisopropylphenyl)-3-(2,4,6-trimethylbenzyl)-1H-imidazol-2-ylidene] dichloropalladium(II);
3-chloropyridine [1-mesityl-3-(2,4,6-trimethylbenzyl)-1H-imidazol-2-ylidene] dichloropalladium(II); and
3-chloropyridine [1-(2,6-diisopropylphenyl)-4,5-dimethyl-3-benzyl-1H-imidazol-2-ylidene] dichloropalladium(II).

1. A compound of formula (I): 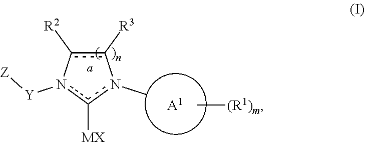
wherein:
M is a group XI (11) transition metal;
X is a counter anion;
Y is C(R^{a})(R^{a});
Z is selected from the group consisting of optionally substituted phenyl, optionally substituted naphthyl, optionally substituted C₄-C₁₀ heteroaryl, —C(═O)C₆-C₁₀ aryl, —C(═O)C₁-C₆ alkyl, —C(═O)OR^{a}, CN, and —C(═O)N(R^{a})(R^{b}),
wherein each optional substituent in Z is independently selected from the group consisting of halogen, hydroxy, amino, NO₂, CN, —C(═O)OR^{a}, N(R^{a})(R^{b}), C₁-C₃ alkyl, C₁-C₃ haloalkyl, C₂-C₆ alkenyl, benzyl, phenyl, and naphthyl, and C₄-C₁₀ heteroaryl;
each occurrence of [symbol] is a single or double bond,
wherein no more than one [symbol] bonding a C atom and a N atom is a double bond;
A¹ is selected from the group consisting of phenyl, naphthyl, and C₄-C₁₀ heteroaryl;
each occurrence of R¹ is independently selected from the group consisting of OR^{a}, N(R^{a})(R^{b}), optionally substituted C₁-C₁₂ alkyl, optionally substituted C₃-C₁₂ cycloalkyl, optionally substituted C₂-C₁₀ heterocycloalkyl, optionally substituted C₂-C₆ alkenyl, optionally substituted benzyl, optionally substituted phenyl, optionally substituted naphthyl, optionally substituted C₄-C₁₀ heteroaryl,
wherein each optional substituent in R¹ is independently selected from the group consisting of C₁-C₃ alkyl, C₂-C₆ alkenyl, benzyl, phenyl, and naphthyl, and C₄-C₁₀ heteroaryl;
R² and R³ are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
R² and R³ may combine with the ring to which they are attached to form a C₄-C₁₂ cycloalkyl, C₆-C₁₀ aryl, or C₄-C₁₀ heteroaryl,
wherein each optional substituent in R² and R³ is independently at least one substituent selected from the group consisting of C₁-C₃ alkyl, C₂-C₆ alkenyl, phenyl, naphthyl, C₄-C₁₀ heteroaryl, OR^{b}, N(R^{a})(R^{b}), CN, CF₃, OCF₃, C(═O)R^{b}, N(R)S(═O)₂R^{b}, C(═O)N(R^{a})(R^{b}), and C(═O)OR^{b};
each occurrence of R^{a} is independently selected from the group consisting of H, optionally substituted C₁-C₃ alkyl, optionally substituted C₃-C₁₂ cycloalkyl, optionally substituted C₂-C₁₀ heterocycloalkyl, optionally substituted C₆-C₁₀ aryl, and optionally substituted C₄-C₁₀ heteroaryl,
wherein each optional substituent in R^{a} is independently at least one substituent selected from the group consisting of C₁-C₃ alkyl, halogen, CN, NO₂, OR^{b}, OCF₃, and CF₃;
each occurrence of R^{b} is independently selected from the group consisting of H, C₁-C₆ alkyl, C₂-C₆ alkenyl, benzyl, phenyl, naphthyl, and C₄-C₁₀ heteroaryl;
m is an integer selected from the group consisting of 0, 1, 2, 3, 4, and 5; and
n is an integer which is either 1 or 2.

2. The compound of claim 1, wherein at least one of the following applies:
(a) Y is —CH₂—;
(b) bond a is a double bond;
(c) m is 2 or 3;
(d) each R¹ is independently selected from the group consisting of methyl, ethyl, n-propyl, isopropyl, n-butyl, sec-butyl, isobutyl, tert-butyl, cyclopropyl, cyclobutyl, cyclopentyl, cyclohexyl, cycloheptyl, cyclooctyl, —CH(CH₂CH₃)₂, —CH(CH(CH₃)₂)₂, —CH(CH₂CH₂CH₂CH₃)₂, phenyl, CHPh₂, —OCH₃, and —O(CH(CH₃)₂); and
(e) n is 1.

3-6. (canceled)

7. The compound of claim 1, which is a compound of formula (II): 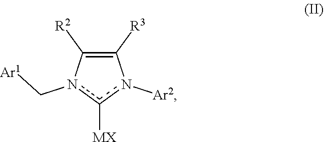
wherein:
X is selected from the group consisting of halogen, OS(═O)₂R^{c}, OC(═O)R^{c}, N(C(═O)R^{c})₂, tetracoordinate boronate, hexacoordinate phosphorus, and H;
each occurrence of R^{c} is independently selected from the group consisting of C₁-C₃ alkyl, C₁-C₃ haloalkyl, and phenyl optionally substituted with at least one substituent selected from the group consisting of C₁-C₃ alkyl and halogen;
A¹ and Ar² are each independently selected from the group consisting of optionally substituted phenyl, optionally substituted naphthyl, and optionally substituted C₄-C₁₀ heteroaryl,
wherein each optional substituent in Ar¹ and Ar² is independently at least one substituent selected from the group consisting of OR^{a}, N(R^{a})(R^{b}), C₁-C₁₂ alkyl, C₃-C₁₂ cycloalkyl, C₂-C₁₀ heterocycloalkyl, C₁-C₃ haloalkyl, benzyl, and —CH₂—(C₆-C₁₀ heteroaryl), and
wherein the optional benzyl and —CH₂—(C₆-C₁₀ heteroaryl) substituents in Ar¹ and Ar² are independently optionally substituted with at least one substituent selected from the group consisting of C₁-C₃ alkyl, C₆-C₁₀ aryl, and C₆-C₁₀ heteroaryl; and
R² and R³ are each independently selected from the group consisting of H, C₁-C₆ alkyl, C₂-C₆ alkenyl, phenyl, and naphthyl, C₄-C₁₀ heteroaryl.

8. The compound of claim 1, wherein at least one of the following applies:
(a) R² and R³ are each independently selected from the group consisting of H, methyl, ethyl, n-propyl, isopropyl, n-butyl, sec-butyl, isobutyl, and tert-butyl;
(b) each of Ar¹ and Ar² is independently selected from the group consisting of phenyl, 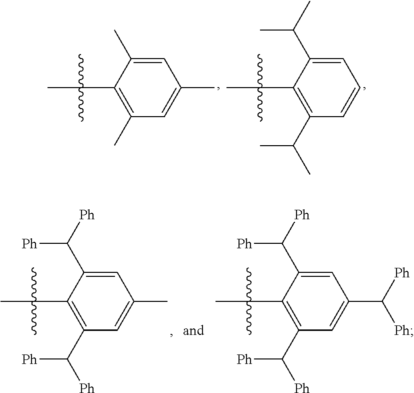
(c) M is selected from the group consisting of Cu, Ag, and Au; and
(d) X is selected from the group consisting of Cl, Br, triflate (OTf), bistriflamide (NTf₂), tosylate (OTs), trifluoroacetate (TFA), BF₄, PF₆, and H.

9. (canceled)

10. The compound of claim 1, wherein R² and R³ are identical.

11-14. (canceled)

15. The compound of claim 1, which is selected from the group consisting of:
1-(2,6-diisopropylphenyl)-4,5-dimethyl-3-(2,4,6-trimethylbenzyl)-imidazol-2-ylidene copper(I) chloride;
1-(2,6-diisopropylphenyl)-3-(2,4,6-trimethylbenzyl)-imidazol-2-ylidene copper(I) chloride;
1-mesityl-4,5-dimethyl-3-(2,4,6-trimethylbenzyl)-imidazol-2-ylidene copper(I) chloride;
1-mesityl-3-(2,4,6-trimethylbenzyl)-imidazol-2-ylidene copper(I) chloride;
1-benzyl-3-(2,6-diisopropylphenyl)-4,5-dimethyl-imidazol-2-ylidene copper(I) chloride;
1-(2,6-diisopropylphenyl)-4,5-dimethyl-3-(2,4,6-trimethylbenzyl)-imidazol-2-ylidene gold(I) bistriflimide;
1-(2,6-diisopropylphenyl)-4,5-dimethyl-3-(2,4,6-trimethylbenzyl)-imidazol-2-ylidene gold(I) chloride;
1-(2,6-diisopropylphenyl)-3-(2,4,6-trimethylbenzyl)-imidazol-2-ylidene gold(I) chloride;
1-mesityl-4,5-dimethyl-3-(2,4,6-trimethylbenzyl)-imidazol-2-ylidene gold(I) chloride;
1-mesityl-3-(2,4,6-trimethylbenzyl)-imidazol-2-ylidene gold(I) chloride;
1-benzyl-3-(2,6-diisopropylphenyl)-4,5-dimethyl-imidazol-2-ylidene gold(I) chloride;
1-(2,6-diisopropylphenyl)-4,5-dimethyl-3-(2,4,6-trimethylbenzyl)-imidazol-2-ylidene silver(I) chloride;
1-(2,6-diisopropylphenyl)-3-(2,4,6-trimethylbenzyl)-imidazol-2-ylidene silver(I) chloride;
1-mesityl-4,5-dimethyl-3-(2,4,6-trimethylbenzyl)-imidazol-2-ylidene silver(I) chloride;
1-mesityl-3-(2,4,6-trimethylbenzyl)-imidazol-2-ylidene silver(I) chloride; and
1-benzyl-3-(2,6-diisopropylphenyl)-4,5-dimethyl-imidazol-2-ylidene silver(I) chloride.

16. A method of promoting a reaction between an alkyne and a borylation reagent, the method comprising contacting the alkyne and the borylation reagent in the presence of a base, a protic solvent or an electrophile, and the compound of any one of claim 1.

17. The method of claim 16, wherein at least one of the following applies:
(a) the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)2, CN, NO₂, CHO, C(═O)OH, C(═O)0(C₁-C₆ alkyl), C(═O)NH₂, C(═O)NH(C₁-C₆ alkyl), and C(═O)N(C₁-C₆ alkyl)2;
(b) the alkyne is a terminal alkyne;
(c) the borylation reagent is a diboronic ester;
(d) the compound is present in an amount ranging from about 0.02 to about 2.0 mol %;
(e) the protic solvent is MeOH;
(f) the base is selected from the group consisting of NaOH and NaOtBu;
(g) the contacting occurs in the presence of an aprotic solvent
(h) the electrophile is selected from the group consisting of C₁-C₁₂ haloalkyl, C₆-C₁₂ haloaralkyl, C₆-C₁₀ aryl halide, C₄-C₁₀ heteroaryl halide, C₁-C₁₂ alkyl-(C═O)Cl, C₆-C₁₀ aryl-(C═O)Cl, C₄-C₁₀ heteroaryl-(C═O)Cl, and CO₂; and
(i) the contacting occurs at a temperature of about 60° C.

18-19. (canceled)

20. The method of claim 17, wherein at least one of the following applies:
(a) the diboronic ester is B₂(pin)₂;
(b) the aprotic solvent is tetrahydrofuran; and
(c) the electrophile is MeI.

21-28. (canceled)

29. A method of promoting hydration of a first reagent, the method comprising contacting the first reagent and water in the presence of the compound of claim 1.

30. The method of claim 29, wherein one of the following applies:
(a) the first reagent comprises an
(b) the contacting occurs in the presence of a Lewis acid;
(c) the compound is present in an concentration of about 100 ppm;
(d) the contacting occurs in the presence of a solvent and
(e) the contacting occurs at a temperature of about 120° C.

31. The method of claim 30, wherein at least one of the following applies:
(a) the alkyne is selected from the group consisting of optionally substituted C₂-C₁₂ alkynyl, optionally substituted C₆-C₁₀ aralkynyl, and optionally substituted C₄-C₁₀ heteroaralkynyl, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)0(C₁-C₆ alkyl), C(═O)NH₂, C(═O)NH(C₁-C₆ alkyl), and C(═O)N(C₁-C₆ alkyl)₂;
(b) the Lewis acid is selected from the group consisting of AgNTf₂, AgOAc, AgOTf, NaBArF, KB(C₆F₅)₄, and AgSbF6;
(c) the solvent is 1,4-dioxane; and
(d) the solvent and water have a ratio of about 2:1.

32-38. (canceled)

39. The method of claim 29, wherein at least one of the following applies:
(a) the first reagent is selected from the group consisting of optionally substituted C₁-C₁₂ alkyl nitrile, optionally substituted C₂-C₆ alkenyl nitrile, optionally substituted C₆-C₁₀ aryl nitrile, optionally substituted C₄-C₁₀ heteroaryl nitrile, wherein each optional substituent is at least one selected from the group consisting of C₁-C₆ alkoxy, C₁-C₆ haloalkoxy, C₁-C₆ haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, halogen, OH, NH₂, NH(C₁-C₆ alkyl), N(C₁-C₆ alkyl)₂, CN, NO₂, CHO, C(═O)OH, C(═O)O(C₁-C₆ alkyl), C(═O)NH₂, C(═O)NH(C₁-C₆ alkyl), and C(═O)N(C₁-C₆ alkyl)₂;
(b) the contacting occurs in the presence of a solvent; and
(c) the contacting occurs at a temperature of about 140° C.

40. (canceled)

41. The method of claim 39, wherein at least one of the following applies:
(a) the solvent is tetrahydrofuran; and
(b) the solvent and water have a ratio of about 1:1.

42-43. (canceled)

44. A compound of formula (III): 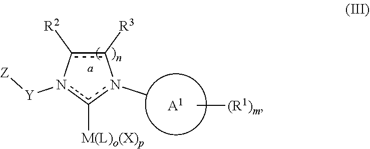
wherein:
M is a group X (10) transition metal;
L is a ligand of M, wherein each occurrence of L can be the same or different;
X is a counter anion;
Y is C(R^{a})(R^{a});
Z is selected from the group consisting of optionally substituted phenyl, optionally substituted naphthyl, optionally substituted C₄-C₁₀ heteroaryl, —C(═O)C₆-C₁₀ aryl, —C(═O)C₁-C₆ alkyl, —C(═O)OR^{a}, CN, and —C(═O)N(R^{a})(R^{b}),
wherein each optional substituent in Z is independently selected from the group consisting of halogen, hydroxy, amino, NO₂, CN, —C(═O)OR^{a}, N(R^{a})(R^{b}), C₁-C₃ alkyl, C₁-C₃ haloalkyl, C₂-C₆ alkenyl, benzyl, phenyl, and naphthyl, and C₄-C₁₀ heteroaryl;
each occurrence of [symbol] is a single or double bond,
wherein no more than one [symbol] bonding a C atom and a N atom is a double bond;
A¹ is selected from the group consisting of phenyl, naphthyl, and C₄-C₁₀ heteroaryl;
each occurrence of R¹ is independently selected from the group consisting of OR^{a}, N(R^{a})(R^{b}), optionally substituted C₁-C₁₂ alkyl, optionally substituted C₃-C₁₂ cycloalkyl, optionally substituted C₂-C₁₀ heterocycloalkyl, optionally substituted C₂-C₆ alkenyl, optionally substituted benzyl, optionally substituted phenyl, optionally substituted naphthyl, optionally substituted C₄-C₁₀ heteroaryl,
wherein each optional substituent in R¹ is independently selected from the group consisting of C₁-C₃ alkyl, C₂-C₆ alkenyl, benzyl, phenyl, and naphthyl, and C₄-C₁₀ heteroaryl;
R² and R³ are each independently selected from the group consisting of H, optionally substituted C₁-C₆ alkyl, optionally substituted C₂-C₆ alkenyl, optionally substituted benzyl, optionally substituted phenyl, and optionally substituted naphthyl, and optionally substituted C₄-C₁₀ heteroaryl, or
R² and R³ may combine with the ring to which they are attached to form a C₄-C₁₂ cycloalkyl, C₆-C₁₀ aryl, or C₄-C₁₀ heteroaryl,
wherein each optional substituent in R² and R³ is independently at least one substituent selected from the group consisting of C₁-C₃ alkyl, C₂-C₆ alkenyl, phenyl, naphthyl, C₄-C₁₀ heteroaryl, OR^{b}, N(R^{a})(R^{b}), CN, CF₃, OCF₃, C(═O)R^{b}, N(R)S(═O)₂R^{b}, C(═O)N(R^{a})(R^{b}), and C(═O)OR^{b};
each occurrence of R^{a} is independently selected from the group consisting of H, optionally substituted C₁-C₃ alkyl, optionally substituted C₃-C₁₂ cycloalkyl, optionally substituted C₂-C₁₀ heterocycloalkyl, optionally substituted C₆-C₁₀ aryl, and optionally substituted C₄-C₁₀ heteroaryl,
wherein each optional substituent in R^{a} is independently at least one substituent selected from the group consisting of C₁-C₃ alkyl, halogen, CN, NO₂, OR^{b}, OCF₃, and CF₃;
each occurrence of R^{b} is independently selected from the group consisting of H, C₁-C₆ alkyl, C₂-C₆ alkenyl, benzyl, phenyl, naphthyl, and C₄-C₁₀ heteroaryl;
m is an integer selected from the group consisting of 0, 1, 2, 3, 4, and 5;
n is an integer which is either 1 or 2;
o is an integer selected from the group consisting of 1 and 2; and
p is an integer selected from the group consisting of 1 and 2.

45. The compound of claim 44, wherein at least one of the following applies:
(a) Y is —CH₂—;
(b) bond a is a double bond;
(c) m is 2 or 3;
(d) n is 1;
(e) o is 1;
(f) p is 2; and
(g) each R¹ is independently selected from the group consisting of methyl, ethyl, n-propyl, isopropyl, n-butyl, sec-butyl, isobutyl, tert-butyl, cyclopropyl, cyclobutyl, cyclopentyl, cyclohexyl, cycloheptyl, cyclooctyl, —CH(CH₂CH₃)₂, —CH(CH(CH₃)₂)₂, —CH(CH₂CH₂CH₂CH₃)₂, phenyl, CHPh₂, —OCH₃, and —O(CH(CH₃)₂).

46-51. (canceled)

52. The compound of claim 44, which is a compound of formula (IV): 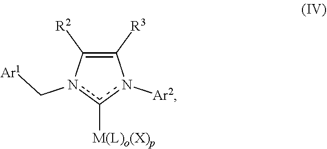
wherein:
X is selected from the group consisting of halogen, OS(═O)₂R^{c}, OC(═O)R^{c}, N(C(═O)R^{c})₂, tetracoordinate boronate, hexacoordinate phosphorus, H, optionally substituted C₆-C₁₀ aryl, nd optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl;
L optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl;
each occurrence of R^{c} is independently selected from the group consisting of C₁-C₃ alkyl, C₁-C₃ haloalkyl, and phenyl optionally substituted with at least one substituent selected from the group consisting of C₁-C₃ alkyl and halogen;
Ar¹ and Ar² are each independently selected from the group consisting of optionally substituted phenyl, optionally substituted naphthyl, and optionally substituted C₄-C₁₀ heteroaryl,
wherein each optional substituent in Ar¹ and Ar² is independently at least one substituent selected from the group consisting of OR^{a}, N(R^{a})(R^{b}), C₁-C₁₂ alkyl, C₃-C₁₂ cycloalkyl, C₂-C₁₀ heterocycloalkyl, C₁-C₃ haloalkyl, benzyl, and —CH₂—(C₆-C₁₀ heteroaryl), and
wherein the optional benzyl and —CH₂—(C₆-C₁₀ heteroaryl) substituents in Ar¹ and Ar² are independently optionally substituted with at least one substituent selected from the group consisting of C₁-C₃ alkyl, C₆-C₁₀ aryl, and C₆-C₁₀ heteroaryl; and
R² and R³ are each independently selected from the group consisting of H, C₁-C₆ alkyl, C₂-C₆ alkenyl, phenyl, and naphthyl, C₄-C₁₀ heteroaryl.

53. The compound of claim 52, wherein at least one of the following applies:
(a) X is selected from the group consisting of Cl, Br, tosylate (OTs), mesylate (OMs), triflate (OTf), bistriflamide (NTf₂), allyl anion (i.e., vinylmethanide), and allylbenzene anion (i.e., 3-phenylpropen-3-ide and/or 1-phenylpropen-3-ide);
(b) L is selected from the group consisting of Cl and 3-chloropyridine;
(c) R² and R³ are each independently selected from the group consisting of H, methyl, ethyl, n-propyl, isopropyl, n-butyl, sec-butyl, isobutyl, and tert-butyl;
(d) Ar¹ and Ar² is independently selected from the group consisting of phenyl, 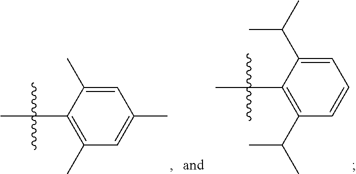
and
(e) M is selected from the group consisting of Pd, Ni, and Pt.

54-56. (canceled)

57. The compound of claim 52, wherein R² and R³ are identical.

58-60. (canceled)

61. The compound of claim 44, which is selected from the group consisting of:
allyl [1-(2,6-diisopropylphenyl)-4,5-dimethyl-3-(2,4,6-trimethylbenzyl)-1H-imidazol-2-ylidene] chloropalladium(II);
cinnamyl [1-(2,6-diisopropylphenyl)-4,5-dimethyl-3-(2,4,6-trimethylbenzyl)-1H-imidazol-2-ylidene] chloropalladium(II);
cinnamyl [1-mesityl-4,5-dimethyl-3-(2,4,6-trimethylbenzyl)-1H-imidazol-2-ylidene] chloropalladium(II);
cinnamyl [1-(2,6-diisopropylphenyl)-3-(2,4,6-trimethylbenzyl)-1H-imidazol-2-ylidene] chloropalladium(II);
cinnamyl [1-mesityl-3-(2,4,6-trimethylbenzyl)-1H-imidazol-2-ylidene] chloropalladium(II);
cinnamyl [1-(2,6-diisopropylphenyl)-4,5-dimethyl-3-benzyl-1H-imidazol-2-ylidene] chloropalladium(II);
3-chloropyridine [1-(2,6-diisopropylphenyl)-4,5-dimethyl-3-(2,4,6-trimethylbenzyl)-1H-imidazol-2-ylidene] dichloropalladium(II);
3-chloropyridine [1-mesityl-4,5-dimethyl-3-(2,4,6-trimethylbenzyl)-1H-imidazol-2-ylidene] dichloropalladium(II);
3-chloropyridine [1-(2,6-diisopropylphenyl)-3-(2,4,6-trimethylbenzyl)-1H-imidazol-2-ylidene] dichloropalladium(II);
3-chloropyridine [1-mesityl-3-(2,4,6-trimethylbenzyl)-1H-imidazol-2-ylidene] dichloropalladium(II); and
3-chloropyridine [1-(2,6-diisopropylphenyl)-4,5-dimethyl-3-benzyl-1H-imidazol-2-ylidene] dichloropalladium(II).
